(19) United States
(12) Patent Application Publication
(10) Pub. No.: US 2026/0035704 A1
(43) Pub. Date: Feb. 5, 2026

(54) COMPOSITIONS AND METHODS INVOLVING APTAMER SWITCH POLYNUCLEOTIDES

(71) Applicants: CZ Biohub SF, LLC, San Francisco, CA (US); The Board of Trustees of the Leland Stanford Junior University, Stanford, CA (US)

(72) Inventors: Hyongsok Tom Soh, Palo Alto, CA (US); Brandon Daniel Wilson, Redwood City, CA (US); Amani Hariri, Stanford, CA (US)

(21) Appl. No.: 19/362,808

(22) Filed: Oct. 20, 2025

Related U.S. Application Data
(62) Division of application No. 17/287,665.
(60) Provisional application No. 62/750,056, filed on Oct. 24, 2018.
(60) Provisional application No. 62/867,783, filed on Jun. 27, 2019.

(51) Int. Cl. C12N 15/115; C12Q 1/6818

(52) U.S. Cl. CPC C12N 15/115; C12Q 1/6818; C12N 2310/16; C12N 2310/3517; C12N 2310/3519

(57) ABSTRACT

The disclosure provides aptamer switch polynucleotides whose kinetics and effective binding affinity to a target analyte can be independently tuned. The aptamer switch polynucleotides comprise an aptamer, an intramolecular linker, and a displacement strand.

### CROSS-REFERENCES TO RELATED APPLICATIONS

This application is a divisional of U.S. National Stage Application No. 17/287,665, filed Apr. 22, 2021, which claims benefit of PCT Application No. PCT/US2019/057726, filed Oct. 23, 2019, which claims benefit to Provisional Application No. 62/750,056, filed Oct. 24, 2018, and to U.S. Provisional Application No. 62/867,783, filed Jun. 27, 2019, the entire contents of which are incorporated herein by reference for all purposes.

### FEDERALLY SPONSORED RESEARCH AND DEVELOPMENT

This invention was made with Government support under contracts OD025342 and GM129313 awarded by the National Institutes of Health. The Government has certain rights in the invention.

### REFERENCE TO AN ELECTRONIC SEQUENCE LISTING

The contents of the electronic sequence listing (110221-1521138-003120US SL.xml; Size: 55,791 bytes; Date of Creation: Oct. 14, 2025) is herein incorporated by reference in its entirety.

### BACKGROUND OF THE INVENTION

Molecular switches that change their conformation upon target analyte binding offer powerful capabilities for biotechnology and synthetic biology.

### BRIEF SUMMARY OF THE INVENTION

In one aspect, the disclosure features an aptamer switch polynucleotide comprising: an aptamer, wherein the aptamer is linked to a first label at a first terminus of the aptamer; a polynucleotide linker, wherein a first terminus of the polynucleotide linker is linked to a second terminus of the aptamer; and a displacement strand, wherein a first terminus of the displacement strand is linked to a second terminus of the polynucleotide linker, wherein a second terminus of the displacement strand is linked to a second label, and wherein the displacement strand is at least partially complementary to a portion of the aptamer, wherein in the concentration of a target analyte causes a shift in equilibrium between a reporting state and a non-reporting state of the aptamer switch polynucleotide, and wherein one of the first and second labels produces a detectable readout in the reporting state of the aptamer switch polynucleotide.

In some embodiments, in the absence of the target analyte, the displacement strand hybridizes to the portion of the aptamer. In some embodiments, in the presence of the target analyte, the aptamer binds to the target analyte, the displacement strand does not hybridize to the portion of the aptamer.

In some embodiments, one of the first and second labels is a fluorophore and the other of the first and second labels is a quencher. In certain embodiments, the first label is a fluorophore and the second label is a quencher. In certain embodiments, the first label is a quencher and the second label is a fluorophore. The quencher can quench the fluorescence from the fluorophore in the non-reporting state of the aptamer switch polynucleotide.

In some embodiments, the fluorophore produces fluorescence as a detectable readout in the reporting state of the aptamer switch polynucleotide.

In certain embodiments, the polynucleotide linker is a homopolymeric polynucleotide, e.g., a poly-thymine polynucleotide.

In some embodiments of this aspect, the displacement strand is between 60% and 100% complementary (e.g., between 80% and 100% complementary or between 95% and 100% complementary) to the portion of the aptamer. In some embodiments, the displacement strand comprises 1 or 2 mismatched nucleotides. In certain embodiments, the displacement strand is between 3 and 15 nucleotides long.

In some embodiments of this aspect, the displacement strand is at least partially complementary to an end or internal sequence of the aptamer.

In some embodiments of this aspect, the polynucleotide linker is between 10 and 100 nucleotides long.

In some embodiments of this aspect, the aptamer switch polynucleotide comprises natural and/or non-natural nucleotides, for example, natural and/or non-natural DNA and/or RNA nucleotides.

In another aspect, the disclosure features a method of adjusting kinetics and/or effective binding affinity of an aptamer switch polynucleotide described herein, the method comprising: (1) generating a plurality of aptamer switch polynucleotides having (i) different displacement strand lengths, (ii) different intramolecular linker (e.g., polynucleotide linker) lengths, or (iii) different displacement strands; and (2) measuring binding of the aptamer switch polynucleotides to a target analyte.

In another aspect, the disclosure features a method of adjusting kinetics and/or effective binding affinity of an aptamer switch polynucleotide, the method comprising: (1) generating an aptamer switch polynucleotide having (i) an aptamer, (ii) an intramolecular linker, and (iii) a displacement strand: (2) measuring binding of the aptamer switch polynucleotide to a target analyte: (3) changing the length of the intramolecular linker, the length of the displacement strand, or the sequence of the displacement strand to introduce one or more mismatched nucleotides: (4) re-measure binding of the aptamer switch polynucleotides to the target analyte: and (5) optionally repeat steps (3) and (4) until the desired kinetics and/or effective binding affinity of the aptamer switch polynucleotide is reached.

In some embodiments of this aspect, the binding is measured by a chemical and/or physical signal produced by the aptamer switch polynucleotide in the presence of the target analyte compared to in the absence of the target analyte. In certain embodiments, the binding is measured by a generation of fluorescence produced by the aptamer switch polynucleotide in the presence of the target analyte compared to in the absence of the target analyte.

In some embodiments of this aspect, the different displacement strands have different degrees of complementarity to a portion of the aptamer.

In some embodiments, the polynucleotide linker or the intramolecular linker is a homopolymeric polynucleotide, e.g., a poly-thymine polynucleotide.

In some embodiments of this aspect, decreasing the length of the polynucleotide linker or the intramolecular linker results in an increase in kinetics of the aptamer switch polynucleotide. In other embodiments, increasing the length of the polynucleotide linker or the intramolecular linker results in a decrease in kinetics of the aptamer switch polynucleotide.

In some embodiments of this aspect, decreasing the length of the displacement strand results in an increase in kinetics of the aptamer switch polynucleotide. In other embodiments, increasing the length of the displacement strand results in a decrease in kinetics of the aptamer switch polynucleotide.

In some embodiments of this aspect, introducing one or more mismatched nucleotides to the displacement strand results in an increase in kinetics of the aptamer switch polynucleotide.

In some embodiments of this aspect, decreasing the length of the polynucleotide linker or the intramolecular linker and decreasing the length of the displacement strand result in an increase in kinetics of the aptamer switch polynucleotide.

In some embodiments of this aspect, decreasing the length of the polynucleotide linker or the intramolecular linker results in a decrease in effective binding affinity of the aptamer to the target analyte. In other embodiments, increasing the length of the polynucleotide linker or the intramolecular linker results in an increase in effective binding affinity of the aptamer to the target analyte.

In some embodiments of this aspect, decreasing the length of the displacement strand results in an increase in effective binding affinity of the aptamer to the target analyte. In other embodiments, increasing the length of the displacement strand results in a decrease in effective binding affinity of the aptamer to the target analyte.

In some embodiments of this aspect, introducing one or more mismatched nucleotides to the displacement strand results in an increase in effective binding affinity of the aptamer to the target analyte.

In some embodiments of this aspect, increasing the length of the polynucleotide linker or the intramolecular linker and decreasing the length of the displacement strand result in an increase in effective binding affinity of the aptamer switch polynucleotide.

In yet another aspect, the disclosure provides a library of different aptamer switch polynucleotides as described herein, wherein the different aptamer switch polynucleotides have (i) different intramolecular linker lengths, (ii) different displacement strand lengths, or (iii) different displacement strands.

In some embodiments of this aspect, the intramolecular linker is a homopolymeric polynucleotide (e.g., a poly-thymine polynucleotide).

In some embodiments of this aspect, the library has at least 2 different aptamer switch polynucleotides.

In some embodiments of this aspect, each displacement strand in an aptamer switch polynucleotide in the library independently comprises one or more mismatched nucleotides.

### BRIEF DESCRIPTION OF THE DRAWINGS

FIG. 1: Overview of intramolecular strand-displacement and relevant parameters. An intramolecular strand-displacement (ISD) design was used to convert an existing aptamer into a switch that gives a concentration-dependent signal based on the interaction of a fluorophore-quencher pair at the 5′-and 3′-ends of the construct, respectively. In this three-state population shift model, signal is generated by the unfolded and target-bound forms. In the absence of target, the quenched and unfolded states are in equilibrium, defined by K_{Q}. Target binding depletes the unfolded population, and the reaction shifts to the right, generating signal that is proportional to target concentration. This model was used to generalize the discussion and insights to other aptamers, however, it is noted that the ATP aptamer has two binding sites. Therefore, a modified two-site binding model was used for fitting and calculations (FIG. 2).

FIG. 2: Two-site binding model. This model is specific to the ATP aptamer.

FIGS. 3A-3E: Raw signal analysis and selectivity of constructs. FIG. 3A: Representative concentration-dependent emission spectra of an ISD switch. The fluorescence at peak emission (570 nm) was used as raw signal for both thermodynamic and kinetic studies. Concentrations of ATP in mM are listed. FIGS. 3B-3E: Constructs with linker and double-stranded regions of varying lengths retain the high selectivity of the native aptamer for 100 μM ATP versus a 10-fold higher concentration of non-target ribonucleotides. All plots are averaged over n=3 replicates. Error bars represent the standard deviation.

FIGS. 4A-4C: Schematic of ISD control parameters. FIG. 4A: By modulating the length of the linker (Lₗₒₒₚ) and the hybridization strength of the double-stranded hairpin (L_{DS}) formed between the displacement strand and the aptamer, both the kinetics and thermodynamics of the ISD switch can be controlled. FIG. 4B: On one hand, reducing L_{DS} increases effective binding affinity at the expense of increased background. On the other hand, decreasing Lₗₒₒₚ decreases background at the expense of lower effective binding affinity. Decreasing either parameter increases overall rate of binding, making it possible to increase the kinetics of a given construct while retaining the same K_{D}^{eff}.

FIGS. 5A and 5B: Overview of results for various ISD constructs used in this work. The constructs tested are graphically summarized (FIG. 5A). The table (FIG. 5B) summarizes all thermodynamic and kinetic parameters for these constructs. For constructs where LDS=5, kinetics were faster than the time resolution of the detector, and thus, a robust fit was not obtained.

FIG. 6: Sequences used in this work. Black represents the aptamer sequence, blue represents the poly-T linker, and orange represents the displacement strand. Underlined sequence is complementary to the displacement strand. Lₗₒₒₚ is equal to the length of the linker plus the number of bases between the underlined region and the displacement strand. The loop length in this design cannot be shorter than that of the aptamer minus the length of the displacement strand (in this case, 23 nt). Mismatches introduced into the ISD constructs are shown in red.

FIG. 7: Conformational selection model. It has been suggested that the ATP aptamer used in this work can undergo both induced-fit and conformational selection binding mechanisms. Therefore the equations governing ISD are represented through a single-site, conformational selection binding mechanism. Since the pre-folded, binding-competent form also gives a signal, aptamers exhibiting this binding mechanism are more likely to suffer from larger background signal or possibly slower kinetics.

FIGS. 8A-8E: Binding curve modulation via ISD switch design. FIG. 8A: Effects of changing L_{DS} from 5 to 9 nt while maintaining Lₗₒₒₚ at 33 nt. FIG. 8B: K_{d}^{eff} as a function of L_{DS} given a fixed Lₗₒₒₚ. FIG. 8C: Effects of changing Lₗₒₒₚ from 23 to 43 nt while L_{DS} is held constant at 7 bp. FIG. 8D: K_{d}^{eff} as a function of L_{DS} at different Lₗₒₒₚ. FIG. 8E: The removal of a single base from the displacement strand of 8_23, generating 7_23, caused the same binding curve shift as adding 20 bases to the linker of 8_23, generating 8_43. All plots are averaged from three replicates. Error bars in FIGS. 8A, 8C, and 8E represent the standard deviation of the average; error bars in FIGS. 8B and 8D represent the standard deviation of individual fits to each replicate (FIG. 2). FIGS. 8A and 8C show raw fluorescence data, whereas FIG. 8E is normalized by a single site hyperbolic binding curve. Fit parameters for constructs in which L_{DS}=5 have been omitted from FIGS. 8B and 8D because it was difficult obtain robust fits for the parameters. Raw data for all ISD constructs is provided in FIGS. 9A-9J.

FIGS. 9A-9J: Binding curves for all combinations of displacement strand and loop lengths.

FIGS. 10A-10E: Modulating temporal binding response of the ISD switch construct. FIG. 10A: Normalized signal change upon injection of 1 mM ATP at 3.5 s. Increasing L_{DS} while keeping Lₗₒₒₚ constant at 33 nt results in slower kinetics. FIG. 10B: Effect on k_{obs} of increasing Lₗₒₒₚ in an ISD construct with constant L_{DS} in the presence of 1 mM ATP. FIG. 10C: Normalized signal change upon injection with 1 mM ATP at 3.5 s. Increasing Lₗₒₒₚ while keeping L_{DS} constant at 9 results in slower kinetics. FIG. 10D: Effect on k_{obs} of increasing L_{DS} in an ISD construct with constant Lₗₒₒₚ in the presence of 1 mM ATP. FIG. 10E: k_{obs} as a function of both L_{DS} and Lₗₒₒₚ in the presence of 1 mM ATP. Sequences with L_{DS}=5 had switching responses faster than the time resolution of the detector and could not be fit accurately and have been omitted from FIGS. 10B and 10D. Error bars in FIGS. 10A and 10C represent standard deviation over three replicates, whereas those in FIGS. 10B and 10D represent the 95% confidence intervals in the fit parameter. Confidence intervals for e are listed in FIG. 2.

FIGS. 11A-11C: Independent tuning of kinetics and thermodynamics through simultaneous changes to L_{DS} and Lₗₒₒₚ. FIG. 11A: Three pairs of ISD constructs (I, II, III) were tested. FIG. 11B: All three exhibit nearly identical binding curves, but have been engineered in terms of L_{DS} and Lₗₒₒₚ to exhibit vastly different kinetic responses FIG. 11C. Increasing both L_{DS} and Lₗₒₒₚ simultaneously has synergistic effects on temporal resolution. For example, in pair I, decreasing Lₗₒₒₚ from 36-to 23-nt increases

$${k}_{on}^{DS}$$

while decreasing L_{DS} from 9-to 8-bp increases

$${k}_{off}^{DS} ,$$

resulting in a roughly constant K_{Q} but much more rapid observed kinetics. The

$${K}_{D}^{eff}$$

listed in A and the binding curve fits in B are respectively derived from and normalized to a single hyperbolic binding curve. For kinetics measurements, pair I was run at [ATP]=2.5 mM and pairs II and III were run at [ATP]=1 mM.

FIGS. 12A-12C: Effects of mismatch incorporation. FIG. 12A: When considering perfectly matched, unlinked displacement strands (black circles) the set of theoretically obtainable K_{D}^{DS} features very large jumps (20.6±8.7 per base). Mutation position is defined from the 3′-end of the displacement strand. On the other hand, incorporating single mismatches (colored symbols) greatly decreases this spacing to 1.6±0.8. Calculations were performed using ΔG_{fold} from mfold at room temperature and 6 mM Mg²⁺. FIG. 12B: Measured effective binding affinities for perfectly matched strands (black circles) and mismatches (colored symbols) with Lₗₒₒₚ=33 nt. Adding mismatches greatly increases the tunability of the thermodynamics. FIG. 12C: Introducing mismatches greatly increases the signaling kinetics of the constructs, e.g., yellow vs. black star and orange vs. black diamond. Even a 10 mer DS with mismatches exhibits kinetics orders of magnitude faster than that of the equivalent aptamer beacon (red).

FIGS. 13A and 13B: Fine tuning of binding curves via the incorporation of mismatches. FIG. 13A: Modulating affinity via L_{DS} alone leads to huge jumps in affinity. FIG. 13B: In contrast, the incorporation of single mismatches into the displacement strand produces more granular shifts in effective binding affinity. Mismatches are described such that “9_33 p6CG” indicates that position 6, as defined from the 3′-end, was changed from C to G. Curves are averaged over three replicates and fit to and normalized by a single-site hyperbolic binding curve.

### DETAILED DESCRIPTION OF THE INVENTION

### I. Introduction

Aptamers have proven useful as molecular because they offer excellent binding properties, undergo reversible folding, and can be readily engineered into a wide range of nanostructures. Unfortunately, the thermodynamic and kinetic properties of the aptamer switches developed to date are intrinsically coupled, such that high temporal resolution (switching time) can only be achieved at the cost of lower sensitivity. The disclosure provides aptamer switch polynucleotides whose kinetics and effective binding affinity can be independently tuned. The aptamer switch polynucleotides comprise an aptamer, an intramolecular linker, and a displacement strand. The disclosure also describes in detail further herein a general design strategy including changing the length of the intramolecular linker, the length of the displacement strand, and the sequence of the displacement strand to adjust the kinetics and effective binding affinity of the aptamer switch polynucleotides. The experiments further demonstrate using the design strategy to generate an array of aptamer switch polynucleotides with effective dissociation constants (K_{D}) ranging from 10 μM to 40 mM and binding kinetics ranging from 170 ms to 3 s. The strategy should be broadly applicable to other aptamers switch polynucleotides, enabling the efficient development of switches with characteristics suitable for diverse applications.

### II. Definitions

As used herein, the term “aptamer switch polynucleotide” refers to a polynucleotide that changes its conformation upon binding to a target analyte. An aptamer switch polynucleotide can contain three portions: an aptamer, an intramolecular linker, and a displacement strand. In some embodiments, an aptamer switch polynucleotide can have between 25 and 250 nucleotides. As described in detail herein, the kinetics and effective binding affinity of an aptamer switch polynucleotide can be independently tuned by changing the length of the intramolecular linker, the length of the displacement strand, and/or the sequence of the displacement strand to include one or more mismatched nucleotides.

As used herein, the term “aptamer” refers to an oligonucleotide that can recognize and bind to a target analyte. An aptamer can be selected from an in vitro selection, such as a bead-based selection with flow cytometry or a high-density aptamer array. An aptamer can have between 5 and 175 nucleotides.

As used herein, the term “intramolecular linker” refers to a portion within an aptamer switch polynucleotide that links the aptamer to the displacement strand. The intramolecular linker can provide flexibility to the conformation of the aptamer switch polynucleotide. The intramolecular linker does not interact or hybridize with the aptamer portion or displacement strand portion of the aptamer switch polynucleotide. The intramolecular linker can be a polymer containing one or more types of monomers. In one embodiment, the intramolecular linker can be a small organic molecule, such as polyethylene glycol (PEG) or polypropylene glycol (PPG). In another embodiment, the intramolecular linker can be a polynucleotide linker. In some embodiments, the intramolecular linker is a homopolymeric polynucleotide, which can contain monomeric units of nucleotides (e.g., poly-thymine nucleotides). In other embodiments, an intramolecular linker can contain two or more types of nucleotides. In vet other embodiments, an intramolecular linker can simply be a bond (e.g., a covalent bond) or a linkage between the aptamer and the displacement strand in an aptamer switch polynucleotide. For example, an intramolecular linker can be an internucleoside linkage, such as a phosphate linkage, also referred to as a 3′ to 5′ phosphodiester linkage. The length of the intramolecular linker can serve as a control parameter to modify the kinetics and effective binding affinity of the aptamer switch polynucleotide to a target analyte. In some embodiments, an intramolecular linker can have at least 1 unit (e.g., at least 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 units; e.g., between 10 and 100, between 10 and 90, between 10 and 80, between 10 and 70, between 10 and 60, between 10 and 50, between 10 and 40, between 10 and 30, between 10 and 20, between 20 and 100, between 30 and 100, between 40 and 100, between 50 and 100, between 60 and 100, between 70 and 100, between 80 and 100, or between 90 and 100 units).

As used herein, the term “displacement strand” refers to a portion within an aptamer switch polynucleotide that can hybridize to a portion of the aptamer in the absence of a target analyte. As described in detail further herein, the degree of complementarity between the displacement strand and the portion of the aptamer where the displacement strand hybridizes can be a control parameter to modify the kinetics and effective binding affinity of the aptamer switch polynucleotide to the target analyte. In some embodiments, a displacement strand can hybridize to an end sequence within the aptamer. In other embodiments, a displacement strand can hybridize to an internal sequence within the aptamer. A displacement strand can have between 3 and 15 nucleotides.

As used herein, the terms “first terminus” and “second terminus” refer to the two ends of a polynucleotide or the two ends of a bond (e.g., a covalent bond, an internucleoside linkage, such as a phosphate linkage (also referred to as a 3′ to 5′ phosphodiester linkage)). For example, the two ends of the aptamer in an aptamer switch polynucleotide, the two ends of the intramolecular linker that has at least 1 unit (e.g., at least one nucleotide), or the two ends of the displacement strand can be referred to as the first terminus and the second terminus. In some embodiments, the first terminus refers to the 5′ end of a polynucleotide and the second terminus refers to the 3′ end of the polynucleotide. In some embodiments, the first terminus refers to the 3′ end of a polynucleotide and the second terminus refers to the 5′ end of the polynucleotide. In some embodiments, in the case of a phosphate linkage, the first terminus refers to the end that is connected to the 3′ carbon of a nucleotide and the second terminus refers to the end that is connected to the 5′ carbon of a nucleotide. In other embodiments, in the case of a phosphate linkage, the first terminus refers to the end that is connected to the 5′ carbon of a nucleotide and the second terminus refers to the end that is connected to the 3′ carbon of a nucleotide.

As used herein, the term “fluorophore” refers to a compound, e.g., a small molecule or a protein, which when excited by exposure to a particular wavelength of light, emits light at a different wavelength. Fluorophores can be characterized in terms of their emission profile, or “color.” For example, green fluorophores (e.g., green fluorescent protein (GFP), Cy3, FITC, and Oregon Green) are generally characterized by their emission at wavelengths in the range of 510-550 nm. Red fluorophores (e.g., red fluorescent protein (RFP), Texas Red, Cy5, and tetramethylrhodamine) are generally characterized by their emission at wavelengths in the range of 590-690 nm.

As used herein, the term “quencher” refers to a compound that is capable of reducing or absorbing the emission from a fluorophore. Quenching may occur by any of several mechanisms, including fluorescence resonance energy transfer, photo-induced electron transfer, paramagnetic enhancement of intersystem crossing, Dexter exchange coupling, and excitation coupling, such as the formation of dark complexes. One example of a quencher is a dark quencher, which can absorb excitation energy from a fluorophore and dissipates the energy as heat. Another example of a quencher is a fluorescent quencher, which can absorb excitation energy from a fluorophore and reemit this energy as light.

As used herein, the term “target analyte” refers to a molecule that can be recognized and bound by the aptamer in the aptamer switch polynucleotide. A target analyte can be a small molecule (e.g., a small organic molecule), a protein, a peptide, or a nucleic acid (e.g., DNA or RNA).

As used herein, the term “complementary” or “complementarity” refers to the capacity for base pairing via Watson-Crick hydrogen bonding interactions between nucleobases, nucleosides, or nucleotides of a displacement strand to the nucleobases, nucleosides, or nucleotides at the corresponding positions of the aptamer. In some embodiments, the displacement strand can have complete complementarity to a portion of the aptamer (e.g., an end or internal sequence of the aptamer), which means that all of the nucleotides in the displacement strand are complementary to the nucleotides at the corresponding positions of the aptamer. In some embodiments, the displacement strand can have partial complementarity to a portion of the aptamer (e.g., an end or internal sequence of the aptamer), which means that at least one of the nucleotides in the displacement strand does not form Watson-Crick hydrogen bonding with the nucleotide at the corresponding position of the aptamer. A “degree of complementarity” or “% complementarity” refers to a percentage of nucleobases, nucleosides, or nucleotides of a displacement strand that form Watson-Crick hydrogen bonding interactions with the nucleobases, nucleosides, or nucleotides at the corresponding positions of the aptamer. A “perfect degree of complementarity” or “100% complementarity” refers to that all of the of nucleobases, nucleosides, or nucleotides of a displacement strand form Watson-Crick hydrogen bonding interactions with the nucleobases, nucleosides, or nucleotides at the corresponding positions of the aptamer.

As used herein, the term “mismatched nucleotide” refers to a nucleotide at a specific position in the displacement strand that does not engage in Watson-Crick base pairing with a nucleotide at the corresponding position in the aptamer when the displacement strand hybridizes to a portion of the aptamer.

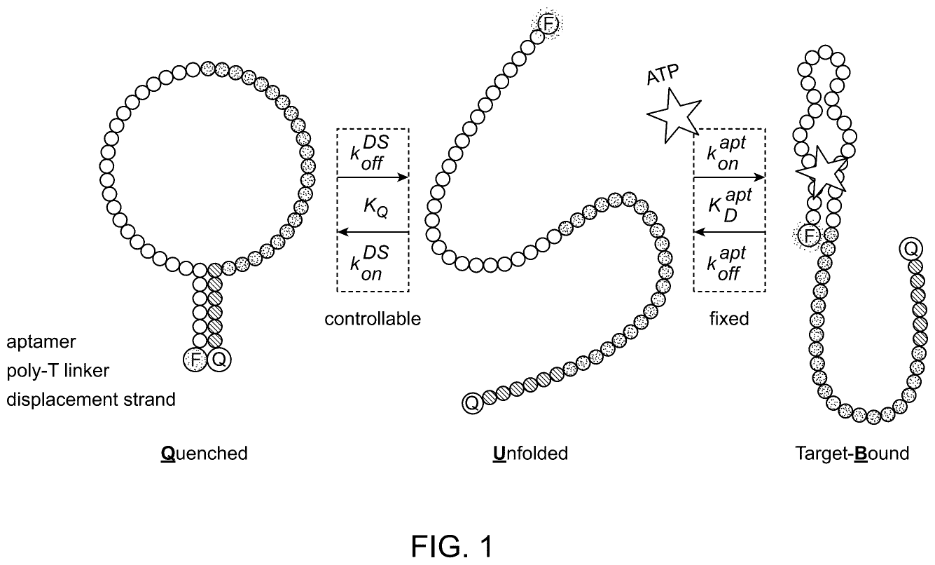

As used herein, the term “kinetics” refers to the temporal resolution of an aptamer switch polynucleotide when it binds to the target analyte, i.e., how long it takes to re-establish equilibrium upon changing target analyte concentration (see, e.g., FIG. 1). As disclosed in detail further herein, changing the length of an intramolecular linker, the length of a displacement strand, or the sequence of a displacement strand can change the kinetics or temporal resolution of the binding between aptamer switch polynucleotide and a target analyte.

As used herein, the term “effective binding affinity” refers to the strength of the non-covalent interaction between two molecules, e.g., an aptamer switch polynucleotide and a target analyte. Effective binding affinity may be quantified by measuring an equilibrium dissociation constant (K_{D}), which refers to the dissociation rate constant (kd, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). Effective binding affinity differs from binding affinity which refers to the intrinsic binding affinity between an aptamer and its target analyte when the aptamer is not within an aptamer switch polynucleotide.

### III. Aptamer Switch Polynucleotides

The disclosure provides aptamer switch polynucleotides that can change their conformation upon binding to a target analyte and whose features can be modified to independently fine tune the kinetics and effective binding affinity of the aptamer switch polynucleotides when binding to the target analyte. The aptamer switch polynucleotides contain certain features that can serve as control parameters that allow one to decouple the kinetics and effective binding affinity of the aptamer switch polynucleotides. For example, by altering one or more of the control parameters, one can increase or decrease the kinetics of the aptamer switch polynucleotides without affecting the effective binding affinity of the aptamer switch polynucleotides. The ability to independently fine tune these properties of the aptamer switch polynucleotides enables the efficient development of aptamer switch polynucleotides that can be used in diverse applications.

An aptamer switch polynucleotide can include: an aptamer, wherein the aptamer is linked to a first label at a first terminus of the aptamer; an intramolecular linker, wherein a first terminus of the intramolecular linker is linked to a second terminus of the aptamer; and a displacement strand, wherein a first terminus of the displacement strand is linked to a second terminus of the intramolecular linker, wherein a second terminus of the displacement strand is linked to a second label, and wherein the displacement strand is at least partially complementary (i.e., comprises one or more mismatched nucleotides) to a portion of aptamer. In some embodiments, one of the first and second labels is a fluorophore and the other of the first and second labels is a quencher. In certain embodiments, the first label is a fluorophore and the second label is a quencher. In other embodiments, the first label is a quencher and the second label is a fluorophore.

The length of the intramolecular linker, the length of the displacement strand, and the degree of complementarity between the displacement strand to the portion of the aptamer can serve as control parameters that enable the independent tuning of the kinetic and effective binding affinity properties of the aptamer switch polynucleotide. By adjusting these control parameters, the conformation of the aptamer switch polynucleotide can be changed based on the effective local concentrations of the target analyte and the aptamer in the aptamer switch polynucleotide, which cause a concentration-dependent shift in equilibrium (see, e.g., FIG. 1). The concentration of a target analyte can cause a shift in equilibrium between a reporting state and a non-reporting state of the aptamer switch polynucleotide. In some embodiments, in the absence of a target analyte, the displacement strand hybridizes to the portion of the aptamer and the quencher quenches the fluorescence from the fluorophore. In the presence of the target analyte, the aptamer binds to the target analyte, the displacement strand does not hybridize to the portion of the aptamer, and the fluorophore produces fluorescence as a detectable readout.

In some embodiments, an aptamer switch polynucleotide of the present disclosure can contain, from 5′ terminus to 3′ terminus, an aptamer, an intramolecular linker, and a displacement strand. In other embodiments, an aptamer switch polynucleotide of the present disclosure can contain, from 5′ terminus to 3′ terminus, a displacement strand, an intramolecular linker, and an aptamer.

A library of different aptamer switch polynucleotides can also be constructed, in which the different aptamer switch polynucleotides have (i) different intramolecular linker lengths, (ii) different displacement strand lengths, or (iii) different displacement strands. These different aptamer switch polynucleotides can have different kinetics and effective binding affinities. In some embodiments, the library has at least 2 (e.g., at least 3, 4, 5, 6, 7, 8, 9, 10, 15, 20, 25, 30, 35, 40, 45, 50, 75, 100, 125, 150, 175, or 200) different aptamer switch polynucleotides.

### Aptamer

An aptamer is a portion of the aptamer switch polynucleotide that can recognize and bind to a target analyte. In the absence of a target analyte, a portion of the aptamer can hybridize to the displacement strand of the aptamer switch polynucleotide. An aptamer can be selected from an in vitro selection, such as a bead-based selection with flow cytometry or a high-density aptamer array. Various aptamers are known and described in the art, see, e.g., International Patent Publication Nos. WO 2014068553 and WO 2016018934, and US Patent Publication No. US 20120263651. In some embodiments, an aptamer can have between 5 and 175 nucleotides (e.g., between 10 and 175, between 20 and 175, between 40 and 175, between 60 and 175, between 80 and 175, between 100 and 175, between 120 and 175, between 140 and 175, between 160 and 175, between 170 and 175, between 5 and 170, between 5 and 160, between 5 and 140, between 5 and 120, between 5 and 100, between 5 and 80, between 5 and 60, between 5 and 40, between 5 and 20, between 5 and 10, 5, 10, 20, 30, 40, 50, 60, 70, 80, 90, 100, 110, 120, 130, 140, 150, 160, 170, or 175 nucleotides).

An aptamer has an intrinsic binding affinity to its target analyte, which refers to the binding affinity between the aptamer and its target analyte when the aptamer is not placed within an aptamer switch polynucleotide. The intrinsic binding affinity between an aptamer and its target analyte can be changed when the aptamer is placed within an aptamer switch polynucleotides. This effective binding affinity refers to the strength of the non-covalent interaction between an aptamer switch polynucleotide and a target analyte. Effective binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As described further herein, the effective binding affinity can be modified by changing the length of the intramolecular linker (e.g., the length of the homopolymeric polynucleotide), the length of the displacement strand, and/or the degree of complementarity between the displacement strand and the portion of the aptamer.

### Intramolecular Linker

An aptamer switch polynucleotide contains an intramolecular linker that links the aptamer portion of the aptamer switch polynucleotide to the displacement strand portion. The intramolecular linker can provide flexibility to the conformation of the aptamer switch polynucleotide. The intramolecular linker does not interact or hybridize with the aptamer or displacement strand portion of the aptamer switch polynucleotide. As demonstrated herein, modifying the length of the intramolecular linker can tune the kinetics and/or the effective binding affinity properties of the aptamer switch polynucleotide. The intramolecular linker can be a polymer containing one or more types of monomers. In one embodiment, the intramolecular linker can be a small organic molecule, such as polyethylene glycol (PEG) or polypropylene glycol (PPG). In another embodiment, the intramolecular linker can be a polynucleotide linker. An intramolecular linker can have at least 1 unit (e.g., at least 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 units; e.g., between 10 and 100, between 10 and 90, between 10 and 80, between 10 and 70, between 10 and 60, between 10 and 50, between 10 and 40, between 10 and 30, between 10 and 20, between 20 and 100, between 30 and 100, between 40 and 100, between 50 and 100, between 60 and 100, between 70 and 100, between 80 and 100, or between 90 and 100 units).

In some embodiments, an intramolecular linker is a homopolymeric polynucleotide. An intramolecular linker can have at least 1 nucleotide (e.g., at least 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 nucleotides; e.g., between 10 and 100, between 10 and 90, between 10 and 80, between 10 and 70, between 10 and 60, between 10 and 50, between 10 and 40, between 10 and 30, between 10 and 20, between 20 and 100, between 30 and 100, between 40 and 100, between 50 and 100, between 60 and 100, between 70 and 100, between 80 and 100, or between 90 and 100 nucleotides).

In some embodiments, when the intramolecular linker is a homopolymeric polynucleotide, the homopolymeric polynucleotide can contain monomeric units of nucleotides (e.g., poly-thymine, poly-adenine, poly-guanine, poly-cytosine, or poly-uracil nucleotides). In particular embodiments, a homopolymeric polynucleotide contains poly-thymine nucleotides. In other embodiments, an intramolecular linker can contain a mixture of two or more types nucleotides, i.e., a mixture of thymine and adenine nucleotides, a mixture of thymine and guanine nucleotides, a mixture of thymine and cytosine nucleotides, or a mixture of thymine, adenine, and guanine nucleotides.

### Displacement Strand

An aptamer switch polynucleotide contains a displacement strand that, in the absence of a target analyte, can hybridize with complete or partial complementarity to a portion of the aptamer in the aptamer switch polynucleotide. In some embodiments, the sequence and length of the displacement strand in the aptamer switch polynucleotide can be modified to tune the kinetic and effective binding affinity properties of the aptamer switch polynucleotide. The displacement strand can contain one or more mismatched nucleotides (e.g., 1, 2, 3, 4, 5, 6, 7, 8, 9, 10, 1-2, 1-4, 1-6, 1-8, 1-10, or more mismatched nucleotides) when the displacement strand hybridizes to a portion of the aptamer. In some embodiments, all of the nucleotides in a displacement strand that hybridize to nucleotides in a portion of the aptamer can be mismatched nucleotides. In some embodiments, the displacement strand is partially complementary, i.e., between 60% and 99% (e.g., between 63% and 99%, between 65% and 99%, between 67% and 99%, between 69% and 99%, between 71% and 99%, between 73% and 99%, between 75% and 99%, between 77% and 99%, between 79% and 99%, between 81% and 99%, between 83% and 99%, between 85% and 99%, between 87% and 99%, between 89% and 99%, between 91% and 99%, between 93% and 99%, between 95% and 99%, between 97% and 99%, between 60% and 97%, between 60% and 95%, between 60% and 93%, between 60% and 91%, between 60% and 89%, between 60% and 87%, between 60% and 85%, between 60% and 83%, between 60% and 81%, between 60% and 79%, between 60% and 77%, between 60% and 75%, between 60% and 73%, between 60% and 71%, between 60% and 69%, between 60% and 67%, between 60% and 65%, between 60% and 63%, between 60% and 61%, 61%, 63%, 65%, 67%, 69%, 71%, 73%, 75%, 77%, 79%, 81%, 83%, 85%, 87%, 89%, 91%, 93%, 95%, 97%, or 99%) complementary to the portion of the aptamer. As described further herein, introducing one or more mismatched nucleotides to the displacement strand of the aptamer switch polynucleotide can significantly increase the kinetics and/or effective binding affinity properties of the aptamer switch polynucleotide when it binds to the target analyte. In other embodiments, the displacement strand can be completely or 100% complementary to the portion of the aptamer.

The length of the displacement strand can be between 3 and 15 nucleotides long (e.g., between 5 and 15, between 7 and 15, between 9 and 15, between 11 and 15, between 13 and 15, between 3 and 13, between 5 and 11, between 5 and 9, between 5 and 7, 3, 4, 5, 6, 7, 8, 9, 10, 11, 12, 13, 14, or 15 nucleotides long). In some embodiments, decreasing the length of the displacement strand can result in an increase in kinetics and/or effective binding affinity properties of the aptamer switch polynucleotide. In other embodiments, increasing the length of the displacement strand can result in a decrease in kinetics and/or effective binding affinity properties of the aptamer switch polynucleotide.

### Labels

An aptamer switch polynucleotide described herein comprises labels that function to produce a detectable readout when the aptamer switch polynucleotide is in a reporting state, i.e., when a target analyte is present. In some embodiments, the labels can produce a chemical and/or physical signal as a detectable readout when the aptamer switch polynucleotide binds to a target analyte.

In some embodiments, the aptamer switch polynucleotide generates fluorescence as a detectable readout when it binds to a target analyte. In this case, one of the first and second labels on the aptamer switch polynucleotide can be a fluorophore and the other of the first and second labels can be a quencher. An aptamer switch polynucleotide can contain a fluorophore at its 5′ (or 3′) terminus and a quencher at its 3′ (or 5′ terminus). In some embodiments, a fluorophore (or quencher) can be conjugated to a terminus of the aptamer portion of the aptamer switch polynucleotide and a quencher (or fluorophore) can be conjugated to a terminus of the displace strand portion of the aptamer switch polynucleotide. When a target analyte is not present, the hybridization between the displacement strand and the portion of the aptamer brings the fluorophore and quench within proximity of each other such that the fluorescence from the fluorophore is quenched by the quencher. When a target analyte is present, the aptamer binds to the target analyte and does not hybridize to the displacement strand. As a result, the fluorophore and the quencher are not within quenching distance of each other and the fluorescence of the fluorophore can serve as a detectable readout for target analyte binding by the aptamer switch polynucleotide.

Examples of fluorophores, as well as quenchers, are known in the art, e.g., as described in Marras, Methods Mol Biol. 335:3-16, 2006; Kozma and Kele, Org Biomol Chem. 17(2):215-233, 2019; and Wang et al., Angew Chem Int Ed Engl. Mar. 7, 2019. Efficient and complete quenching of the fluorescence emitted from the fluorophore by the quencher depends in part on the overlap between the fluorophore emission and quencher absorption spectra. For example, fluorophore coumarin emits at emission wavelength around 472 nm and can be paired with quencher QSY35 which absorbs at wavelength around 475 nm. In another example, fluorophore Alexa 532 emits at emission wavelength around 554 nm and can be a paired with quencher QSY7 which absorbs at wavelength around 560 nm. In yet another example, fluorophore Alexa 647 emits at emission wavelength around 665 nm and can be paired with quencher QSY21 which absorbs at wavelength around 661 nm.

In other embodiments, a label can be a fluorophore whose fluorescence can be quenched when the displacement strand and the portion of the aptamer hybridize to each other in the absence of a target analyte. An example of such a fluorophore is 2-amino purine, whose fluorescence can be quenched when it is stacked with purines and/or pyrimidines (see, e.g., Jean and Hall, Proc Natl Acad Sci U S A. 98(1):37-41, 2001).

In other embodiments, Fluorescence Resonance Energy Transfer (FRET) between a donor fluorophore and an acceptor fluorophore can be used to provide a detectable readout or the absence of a readout. In this case, a first label can be a donor fluorophore and a second label can be an acceptor fluorophore. It is known that, in order for two fluorophores to be FRET partners, the emission spectrum of the donor fluorophore must partially overlap the excitation spectrum of the acceptor fluorophore. In some embodiments, the preferred FRET-partner pairs are those for which the value R0 (Förster distance, distance at which energy transfer is 50% efficient) is greater than or equal to 30 Å. Examples of FRET partners are known in the art, see, e.g., Massey et al., Analytica Chimica Acta 568:181-189, 2006. In some embodiments, when the aptamer switch polynucleotide is in the quenched state in the absence of a target analyte (see, e.g., FIG. 1), the first and second labels are in proximity of each other to produce a FRET signal. The disappearance or reduction of the FRET fluorescence can serve as a signal of target analyte binding. Other fluorescence based methods that can be used to investigate the structure, binding, and dynamics of an aptamer switch polynucleotide can be found in, e.g., Perez-Gonzales et al., Front Chem. 4:33, 2016.

In other embodiments, the labels in the aptamer switch polynucleotide can produce chemical and/or physical signals as a detectable readout when the aptamer switch polynucleotide binds to a target analyte. These signals can be monitored to infer binding to the target analyte. In one example, the labels can be electrochemical reporters (see, e.g., Ferguson et al., Sci Transl Med. 5(213):213ra165, 2013). A first label can be an electrode and a second label can be a redox reporter (e.g., methylene blue). Upon binding to the target analyte, the aptamer switch polynucleotide undergoes a conformational rearrangement that modulates the redox current and generates an electrochemical signal. Other chemical and/or physical signals or techniques that can be used to infer binding of the aptamer switch polynucleotide to a target analyte include, but are not limited to, anisotropy (see, e.g., Gokulrangan et al, Anal Chem. 77(7):1963-70, 2005: Chovelon et al., Biosensors and Bioelectronics 90:140-145, 2017), fluorescence polarization (see, e.g., Perrier et al., Biosensors and Bioelectronics 25:1652-1657, 2010), FETs (field-effect transistors) (see, e.g., Nakatsuka et al., Science 362:319-324, 2018), SERS (surface-enhanced Raman spectroscopy) (see, e.g., Chuong et al., Proc Natl Acad Sci U S A. 114(34):9056, 2017; Sun et al., ACS Appl. Mater. Interfaces 8:5723-5728, 2016; and Lu et al., Analyst 139:3083, 2014).

### IV. Methods

The disclosure also provides methods of adjusting kinetics and/or effective binding affinity of an aptamer switch polynucleotide described herein. Once an aptamer that binds to a target analyte is discovered or already known, the aptamer can be constructed into an aptamer switch polynucleotide, whose kinetics and/or effective binding affinity properties can be modified, by: (1) generating the aptamer switch polynucleotide having (i) an aptamer, (ii) an intramolecular linker, and (iii) a displacement strand; (2) measuring binding of the aptamer switch polynucleotide to the target analyte; (3) changing the length of the intramolecular linker (e.g., the homopolymeric polynucleotide), the length of the displacement strand, and/or the sequence of the displacement strand to introduce one or more mismatched nucleotides; (4) re-measure binding of the aptamer switch polynucleotides to the target analyte; and (5) optionally repeat steps (3) and (4) until the desired kinetics and/or effective binding affinity of the aptamer switch polynucleotide is reached. As described in detail herein, the length of the intramolecular linker in the aptamer switch polynucleotide, the length of the displacement strand in the aptamer switch polynucleotide, and the degree of complementarity between the displacement strand to the portion of the aptamer can serve as control parameters that enable the independent tuning of the kinetic and effective binding affinity properties of the aptamer switch polynucleotide. For example, the effective local concentration of the aptamer in the aptamer switch polynucleotide can be modified by adjusting the length of the intramolecular linker.

In some embodiments, the kinetics of an aptamer switch polynucleotide can be adjusted by decreasing or increasing the length of the intramolecular linker. In some embodiments, decreasing the length of the intramolecular linker can increase the kinetics of the aptamer switch polynucleotide. In some embodiments, increasing the length of the intramolecular linker can decrease the kinetics of the aptamer switch polynucleotide. The length of the intramolecular linker can be adjusted by changing the number of units in the intramolecular linker by one or more at a time. The kinetics of the aptamer switch polynucleotide can be re-measured after each change.

In some embodiments, the kinetics of an aptamer switch polynucleotide can be adjusted by decreasing or increasing the length of the displacement strand. In some embodiments, decreasing the length of the displacement strand can increase the kinetics of the aptamer switch polynucleotide. In some embodiments, increasing the length of the displacement strand can decrease the kinetics of the aptamer switch polynucleotide. The length of the displacement strand can be adjusted by changing the number of nucleotides in the displacement strand by one or more at a time. The kinetics of the aptamer switch polynucleotide can be re-measured after each change.

In some embodiments, the effective binding affinity of an aptamer switch polynucleotide can be adjusted by decreasing or increasing the length of the intramolecular linker. In some embodiments, decreasing the length of the intramolecular linker can decrease the effective binding affinity of the aptamer switch polynucleotide. In some embodiments, increasing the length of the intramolecular linker can increase the effective binding affinity of the aptamer switch polynucleotide. The length of the intramolecular linker can be adjusted by changing the number of nucleotides in the intramolecular linker by one or more at a time. The effective binding affinity of the aptamer switch polynucleotide can be re-measured after each change.

In some embodiments, the effective binding affinity of an aptamer switch polynucleotide can be adjusted by decreasing or increasing the length of the displacement strand. In some embodiments, decreasing the length of the displacement strand can increase the effective binding affinity of the aptamer switch polynucleotide. In some embodiments, increasing the length of the displacement strand can decrease the effective binding affinity of the aptamer switch polynucleotide. The length of the displacement strand can be adjusted by changing the number of nucleotides in the intramolecular linker by one or more at a time. The effective binding affinity of the aptamer switch polynucleotide can be re-measured after each change.

As shown in the Examples, decreasing the length of the intramolecular linker can increase the kinetics and decrease the effective binding affinity of the aptamer switch polynucleotide. Further, the decrease in effective binding affinity can be offset by decreasing the length of the displacement strand, which leads to an overall increase in kinetics. Based on the observed dependencies of these two control parameters, an aptamer switch polynucleotide can be modified to have an increase in kinetics and at the same time, maintain effective binding affinity by decreasing the lengths of the intramolecular linker and the displacement strand simultaneously. Moreover, changing the lengths of the intramolecular linker and the displacement strand can have opposing effects on effective binding affinity of an aptamer switch polynucleotide, but additive effects on the kinetics. Further, the length of the displacement strand can have a more profound impact per base than the length of the intramolecular linker on effective binding affinity.

In further embodiments, the kinetics of an aptamer switch polynucleotide can be adjusted by decreasing or increasing the length of the intramolecular linker and the length of the displacement strand simultaneously. In some embodiments, decreasing both the length of the intramolecular linker and the length of the displacement strand can increase the kinetics of the aptamer switch polynucleotide.

Further, the length of the intramolecular linker and the length of the displacement strand can be changed simultaneously to adjust the effective binding affinity of an aptamer switch polynucleotide. For example, increasing the length of the intramolecular linker and decreasing length of the displacement strand can result in an increase in effective binding affinity of the aptamer switch polynucleotide.

The hybridization strength of the displacement strand to the portion of the aptamer in the aptamer switch polynucleotide can shift the equilibrium away from or toward the quenched state. Specifically, increasing the hybridization strength of the displacement strand to the portion of the aptamer in the aptamer switch polynucleotide by increasing the percent of complementarity between the displacement strand and the portion of the aptamer can shift the equilibrium towards the quenched state, which decreases the background signal but also decreases the effective binding affinity and kinetics. Similarly, decreasing the length of the intramolecular linker also shifts the equilibrium towards the quenched state due to increased effective concentration of the displacement strand. However, decreasing the length of the intramolecular linker also increases the kinetics of the aptamer switch polynucleotide.

In further embodiments, the sequence of the displacement strand can also be used to adjust the kinetics of an aptamer switch polynucleotide as described herein. One or more mismatched nucleotides can be introduced into the displacement strand to increase the kinetics of the aptamer switch polynucleotide. The effect of mismatched nucleotides in a displacement strand on the kinetics of a specific aptamer switch polynucleotide can be tested by first introducing only one mismatched nucleotide into the displacement strand. Additional numbers of mismatched nucleotides can be introduced to further adjust the kinetics of the aptamer switch polynucleotide.

In further embodiments, the sequence of the displacement strand can also be used to adjust the effective binding affinity of an aptamer switch polynucleotide as described herein. One or more mismatched nucleotides can be introduced into the displacement strand to increase the effective binding affinity of the aptamer switch polynucleotide. The effect of mismatched nucleotides in a displacement strand on the effective binding affinity of a specific aptamer switch polynucleotide can be tested by first introducing only one mismatched nucleotide into the displacement strand. Additional numbers of mismatched nucleotides can be introduced to further adjust the effective binding affinity of the aptamer switch polynucleotide.

In order to screen for aptamer switch polynucleotides having the desired kinetics and effective binding affinity properties, a plurality of aptamer switch polynucleotides having (i) different displacement strand lengths, (ii) different intramolecular linker (e.g., homopolymeric polynucleotide) lengths, or (iii) different displacement strands can be generated; and their binding to a target analyte can be measured. The displacement strands in the plurality of aptamer switch polynucleotides can have different lengths and/or difference sequences, in which some displacement strands can have different degrees of complementarity to a portion of the aptamer in the aptamer switch polynucleotide. In some embodiments, a displacement strand can have one or more mismatched nucleotides (e.g., 1, 2, 3, 4, 5, 6, 7, 8, 9, 10, 1-2, 1-4, 1-6, 1-8, 1-10, or more mismatched nucleotides) when the displacement strand hybridizes to a portion of the aptamer. In certain embodiments, the position and/or identity of the mismatched nucleotides can have different effects on the kinetics and effective binding affinity of the aptamer switch polynucleotide. In some embodiments, binding can be measured by a generation of fluorescence from each aptamer switch polynucleotide in the presence of the target analyte compared to in the absence of the target analyte.

### V. Non-Natural Nucleotides

In some embodiments, the aptamer switch polynucleotides can include one or more non-natural nucleotides. A non-natural nucleotide can include one or more of a non-natural nucleobase, a non-natural sugar, and a non-natural internucleoside linkage.

### Non-Natural Nucleobases

A non-natural nucleobase refers to a nucleobase having at least one change that is structurally distinguishable from a naturally-occurring nucleobase (i.e., adenine, guanine, cytosine, thymine, or uracil). In some embodiments, a non-natural nucleobase is functionally interchangeable with its naturally-occurring counterpart. Both naturally-occurring and non-natural nucleobases are capable of hydrogen bonding. Modifications on non-natural nucleobases may help to improve the stability of the aptamer switch polynucleotides to nucleases. In some embodiments, an aptamer switch polynucleotide described herein may include at least one non-natural nucleobase. Examples of non-natural nucleobases include, but are not limited to, 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halouracil, 5-bromouracil, 5-trifluoromethyluracil, 5-halocytosine, 5-bromocytosine, 5-trifluoromethylcytosine, 7-methylguanine, 7-methyladenine, 2-fluoroadenine, 2-aminoadenine, 8-azaguanine, 8-azaadenine, 7-deazaguanine, 7-deazaadenine, 3-deazaguanine, and 3-deazaadenine. In some embodiments, an aptamer switch polynucleotide described herein has one or more non-natural nucleobases (e.g., 5-methylcytosine).

### Non-Natural Sugars

A non-natural sugar refers to a sugar having at least one change that is structurally distinguishable from a naturally-occurring sugar (i.e., 2′-deoxyribose in DNA or ribose in RNA). Modifications on non-natural sugars may help to improve the stability of the aptamer switch polynucleotides to nucleases. In some embodiments, the sugar is a pentofuranosyl sugar. The pentofuranosyl sugar ring of a nucleoside may be non-natural in various ways including, but not limited to, addition of a substituent group, particularly, at the 2′ position of the ring; bridging two non-geminal ring atoms to form a bicyclic sugar (i.e., a locked sugar); and substitution of an atom or group such as —S—, —N(R)— or —C(R₁)(R₂) for the ring oxygen. Examples of non-natural sugars include, but are not limited to, substituted sugars, especially 2′-substituted sugars having a 2′-F, 2′-OCH₂ (2′-OMe), or a 2′—O(CH₂)₂—OCH₃ (2′-O-methoxyethyl or 2′-MOE) substituent group; and bicyclic sugars. A bicyclic sugar refers to a non-natural pentofuranosyl sugar containing two fused rings. For example, a bicyclic sugar may have the 2′ ring carbon of the pentofuranose linked to the 4′ ring carbon by way of one or more carbons (i.e., a methylene) and/or heteroatoms (i.e., sulfur, oxygen, or nitrogen). The second ring in the sugar limits the flexibility of the sugar ring and thus, constrains the oligonucleotide in a conformation that is favorable for base pairing interactions with its target nucleic acids. An example of a bicyclic sugar is a locked sugar, which is a pentofuranosyl sugar having the 2′-oxygen linked to the 4′ ring carbon by way of a carbon (i.e., a methylene) or a heteroatom (i.e., sulfur, oxygen, or nitrogen). In some embodiments, a locked sugar has the 2′-oxygen linked to the 4′ ring carbon by way of a carbon (i.e., a methylene). In other words, a locked sugar has a 4′-(CH2)—O—2′ bridge, such as α-L-methyleneoxy (4′-CH2—O—2′) and β-D-methyleneoxy (4′—CH₂—O—2′). A nucleoside having a lock sugar is referred to as a locked nucleoside.

Other examples of bicyclic sugars include, but are not limited to, (6′S)-6′ methyl bicyclic sugar, aminooxy (4′—CH2—O—N(R)—2′) bicyclic sugar, oxyamino (4′—CH₂—N(R)—O—2′) bicyclic sugar, wherein R is, independently, H, a protecting group or C1-C12 alkyl. The substituent at the 2′ position can also be selected from allyl, amino, azido, thio, O-allyl, O—C1-C10 alkyl, OCF₃, O(CH₂)₂SCH₃, O(CH₂)₂—O—N(Rₘ)(Rₙ), and O—CH₂—C(═O)—N(Rₘ)(Rₙ), wherein each Rₘ and Rₙ is, independently, H or substituted or unsubstituted C1-C10 alkyl.

In some embodiments, a non-natural sugar is an unlocked sugar. An unlocked sugar refers to an acyclic sugar that has a 2′, 3′-seco acyclic structure, where the bond between the 2′ carbon and the 3′ carbon in a pentofuranosyl ring is absent.

### Non-Natural Internucleoside Linkages

An internucleoside linkage refers to the backbone linkage that connects the nucleosides. An internucleoside linkage may be a naturally-occurring internucleoside linkage (i.e., a phosphate linkage, also referred to as a 3′ to 5′ phosphodiester linkage, which is found in DNA and RNA) or a non-natural internucleoside linkage. A non-natural internucleoside linkage refers to an internucleoside linkage having at least one change that is structurally distinguishable from a naturally-occurring internucleoside linkage. Non-natural internucleoside linkages may help to improve the stability of the aptamer switch polynucleotides to nucleases and enhance cellular uptake.

Examples of non-natural internucleoside linkages include, but are not limited to, a phosphorothioate linkage, a phosphorodithioate linkage, a phosphoramidate linkage, a phosphorodiamidate linkage, a thiophosphoramidate linkage, a thiophosphorodiamidate linkage, a phosphoramidate morpholino linkage, and a thiophosphoramidate morpholino linkage, and a thiophosphorodiamidate morpholino linkage, which are known in the art and described in, e.g., Bennett and Swayze, Annu Rev Pharmacol Toxicol. 50:259-293, 2010. A phosphorothioate linkage is a 3′ to 5′ phosphodiester linkage that has a sulfur atom for a non-bridging oxygen in the phosphate backbone of an oligonucleotide. A phosphorodithioate linkage is a 3′ to 5′ phosphodiester linkage that has two sulfur atoms for non-bridging oxygens in the phosphate backbone of an oligonucleotide. A thiophosphoramidate linkage refers to a 3′ to 5′ phospho-linkage that has a sulfur atom for a non-bridging oxygen and a NH group as the 3′-bridging oxygen in the phosphate backbone of an oligonucleotide. In some embodiments, an aptamer switch polynucleotide described herein has at least one phosphorothioate linkage. In some embodiments, all of the internucleoside linkages in an aptamer switch polynucleotide described herein are phosphorothioate linkages.

### EXAMPLES

### Example 1. Aptamer Switch Design and Rationale

A wide range of essential biological functions are governed by the action of molecular ‘switches’^{1,2}, which undergo a reversible conformational change upon binding a specific target molecule. These switches can in turn be coupled to other molecular machinery to trigger a wide range of downstream functions. There is considerable interest in the engineering of biologically inspired molecular switches that can achieve a selective and sensitive output in response to binding a target molecule, which could prove valuable for diverse applications, including imaging^{3,4}, biosensing⁵, and drug delivery^{6,7}. Aptamers have proven to be particularly promising and versatile in this regard⁸ as they are highly stable and easy to synthesize, exhibit reversible binding, and are readily adaptable to chemical modifications. Since conventional methods of aptamer generation do not routinely yield aptamers capable of structure switching, many selection schemes^{9,10} and engineering approaches¹¹⁻¹³ have been developed for the creation of aptamer switches. In contrast to naturally occurring molecular switches that have evolved over millions of years to function under precise physiological conditions, switches based on synthetic affinity reagents must be tuned to match their intended function. Unfortunately, existing selection and engineering strategies offer limited control over the thermodynamic and kinetic properties of the resultant aptamer switches and, by extension, over properties such as effective binding affinity and temporal resolution.

Certain molecular features and parameters can control the binding of molecular switches. For instance, the hybridization strength of the hairpin in a molecular beacon can modulate the effective detection range for target concentrations spanning many orders of magnitude¹⁴. Since this enthalpically driven control is coarse grained, other work has sought fine grained control of effective binding affinity through the entropy change associated with the degree of confinement imposed by a intramolecular linker¹⁵. Previous efforts at tuning the binding properties of aptamer, however, have found that their thermodynamic and kinetic properties are intrinsically coupled¹⁶, such that fast temporal resolution can only be achieved at the cost of either large background signal or lower affinity, or requires high-temperature conditions that can interfere with ligand binding¹⁷. At present, there is no reliable strategy for independently controlling the thermodynamic and kinetic properties of engineered aptamer switches.

Here, the disclosure describes a general framework for the design of aptamer switches that enables independent control over their thermodynamic and kinetic properties. The use of an intramolecular strand-displacement (ISD) strategy¹⁸ and the degree to which the binding properties of this construct can be controlled through rational design were explored. For example, the ISD construct consists of a single-molecule switch in which an aptamer is coupled to a partially complementary displacement strand via a poly-T linker. The key feature of this design is that it offers two distinct control parameters: displacement strand length (L_{DS}) and loop length (Lₗₒₒₚ). The disclosure shows mathematically and experimentally that the two control parameters of the ISD design enabled the precise and independent tuning of the thermodynamics and kinetics of aptamer switches. This approach was used to generate an array of aptamer switches that exhibit affinities ranging over four orders of magnitude, with equilibrium dissociation constants (K_{D}) ranging from 10 μM to 40 mM and binding kinetics ranging from 170 ms to 3 s—all starting from the same parent ATP aptamer^{19,20}. Lastly, it was demonstrated that even tighter control of binding affinity and kinetics can be achieved by introducing single-base mismatches into the displacement strand. This approach should be broadly applicable to virtually any aptamer, thereby enabling wider production of highly controllable aptamer switches that respond to ligands over a wide range of concentrations and time scales.

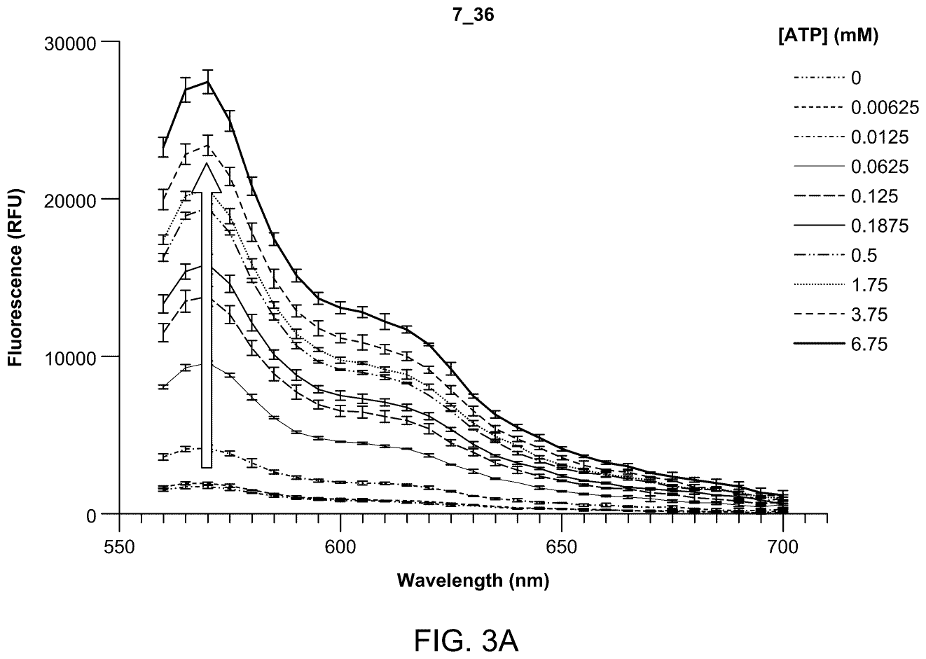

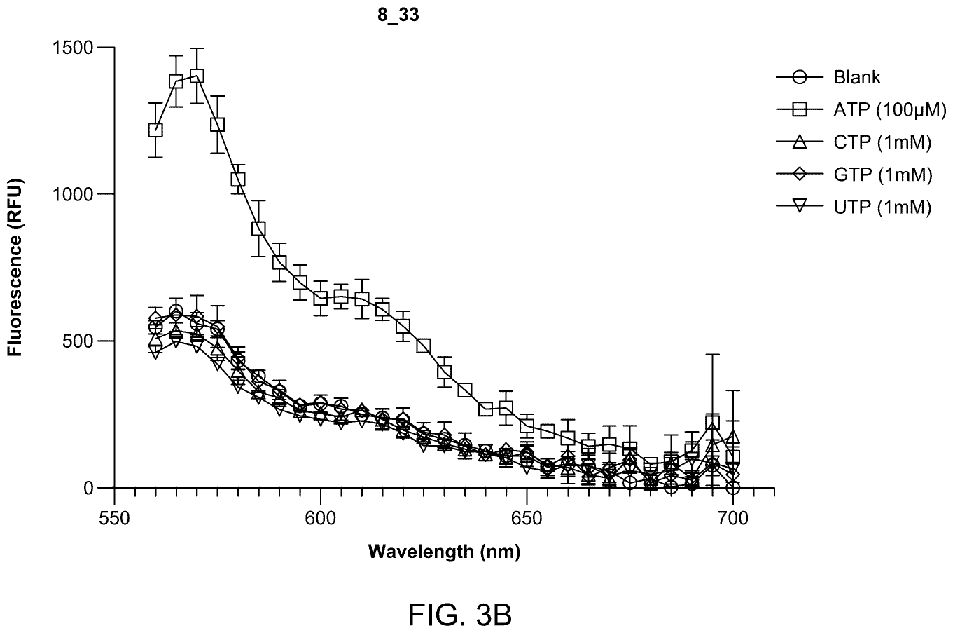

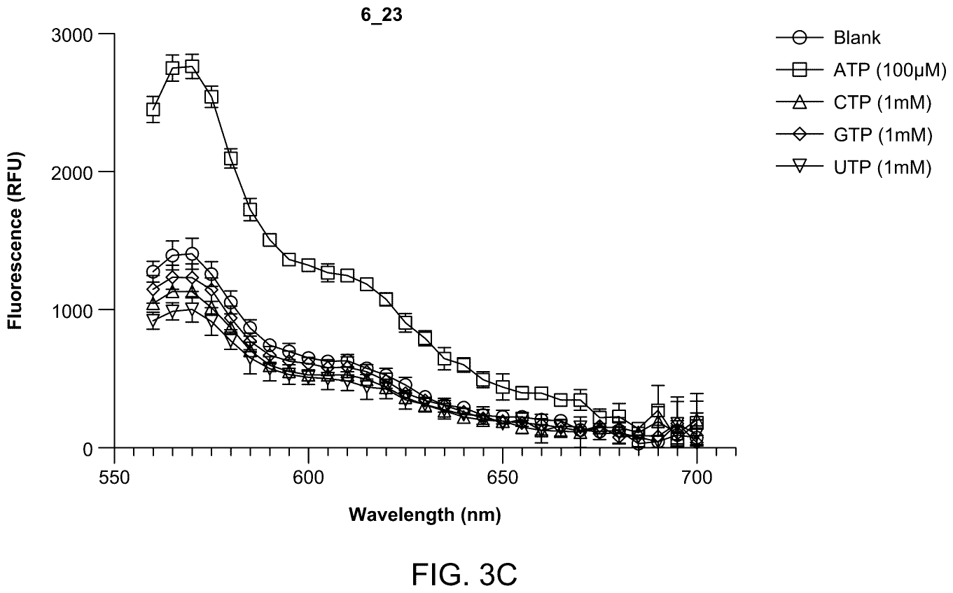

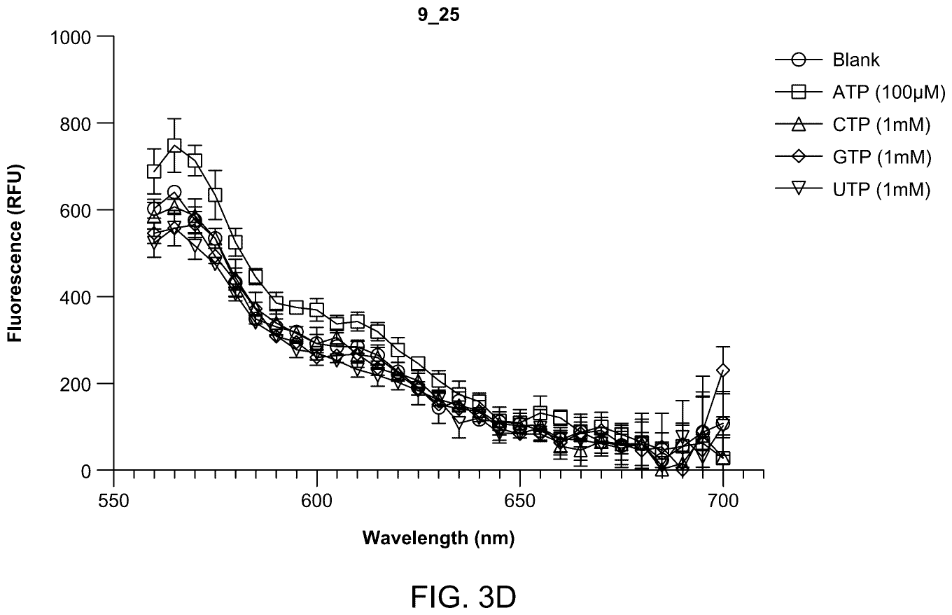

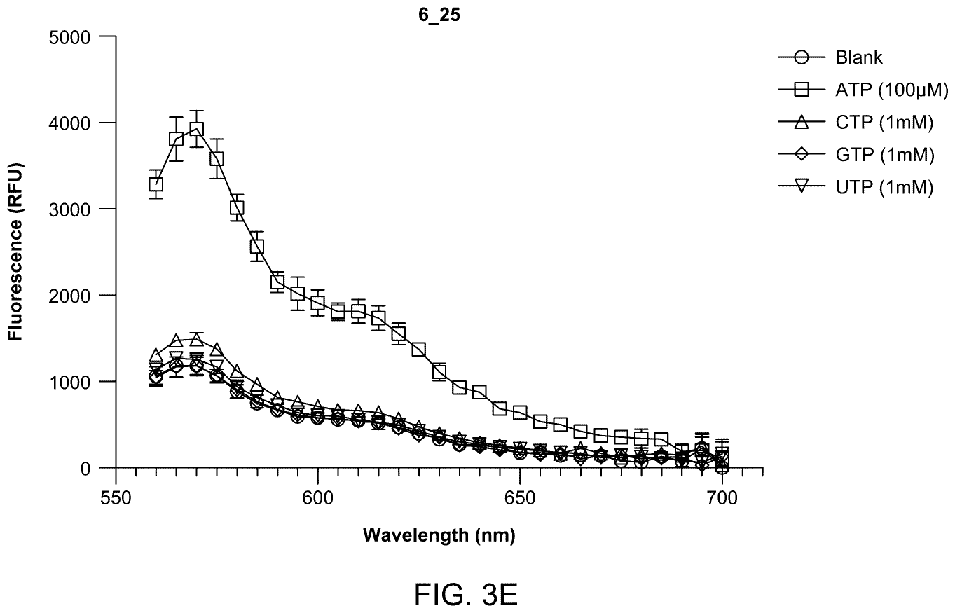

The ISD design enables molecular recognition through concentration-dependent shifts in equilibrium (FIG. 1). As shown in FIG. 1, a fluorophore (F) and a quencher (Q) are added to the 5′-and 3′-ends of the construct, respectively, enabling a fluorescent readout of target concentration (FIG. 3A). Although the thermodynamic and kinetic parameters associated with target binding to the native aptamer

$$( {K}_{D}^{apt} , {k}_{on}^{apt} , {k}_{off}^{apt} )$$

are fixed, the overall signaling response can be tuned by altering the parameters of the hybridization/quenching reaction

$$( {K}_{Q} , {k}_{on}^{DS} , {k}_{off}^{DS} ) .$$

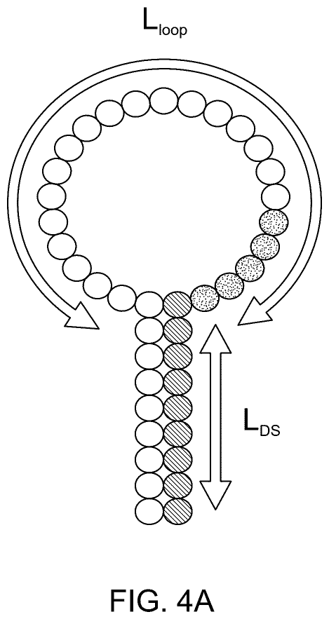

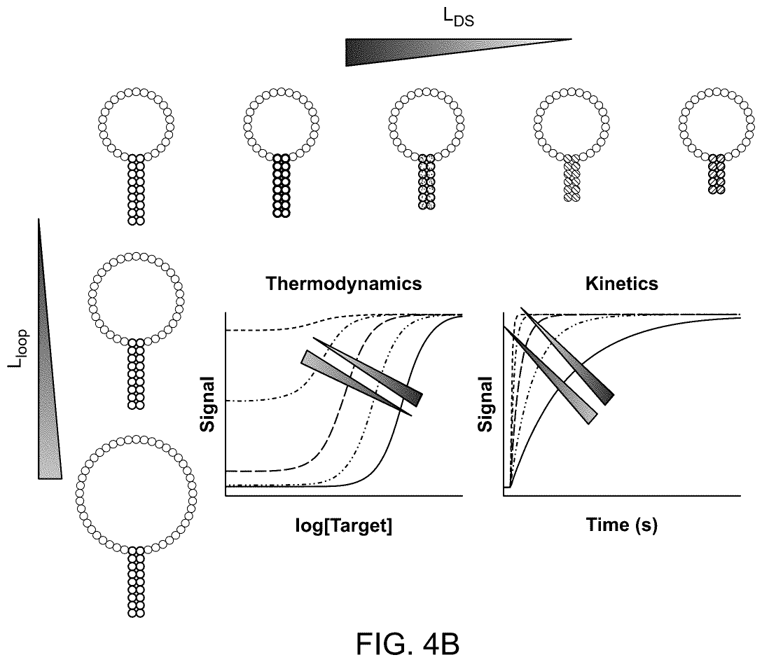

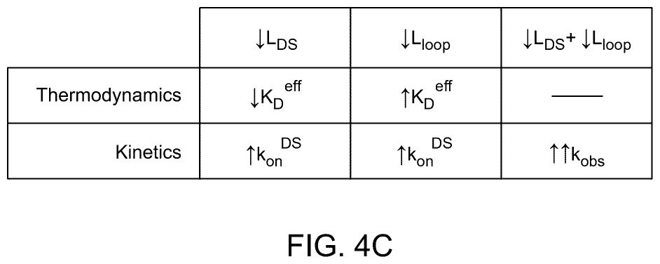

Since hairpin hybridization strength confers coarse-grained control over binding affinity¹⁴, and linker length confers fine-grained control of binding thermodynamics¹⁵, the incorporation of both tuning mechanisms makes this switch design highly amenable to the fine-tuning of molecular recognition (FIG. 4A). Increasing the hybridization strength of the displacement strand shifts the equilibrium towards the quenched state, which will decrease the background signal at the expense of decreased effective affinity and temporal resolution. Decreasing Lₗₒₒₚ results in a similar equilibrium shift due to increased effective concentration of the displacement strand but now with increased temporal resolution. Independent tuning of the ISD's thermodynamics and kinetics is made possible by the orthogonal effects of these two parameters.

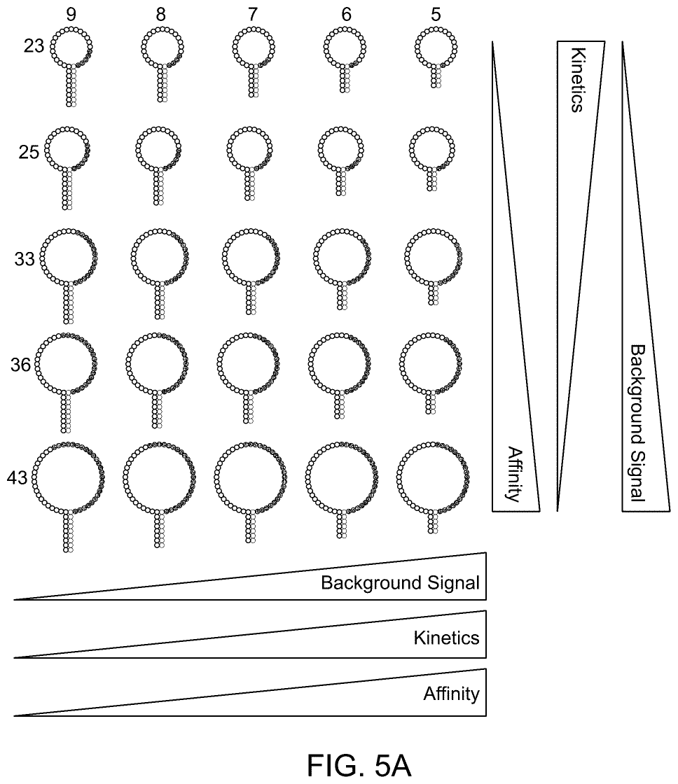

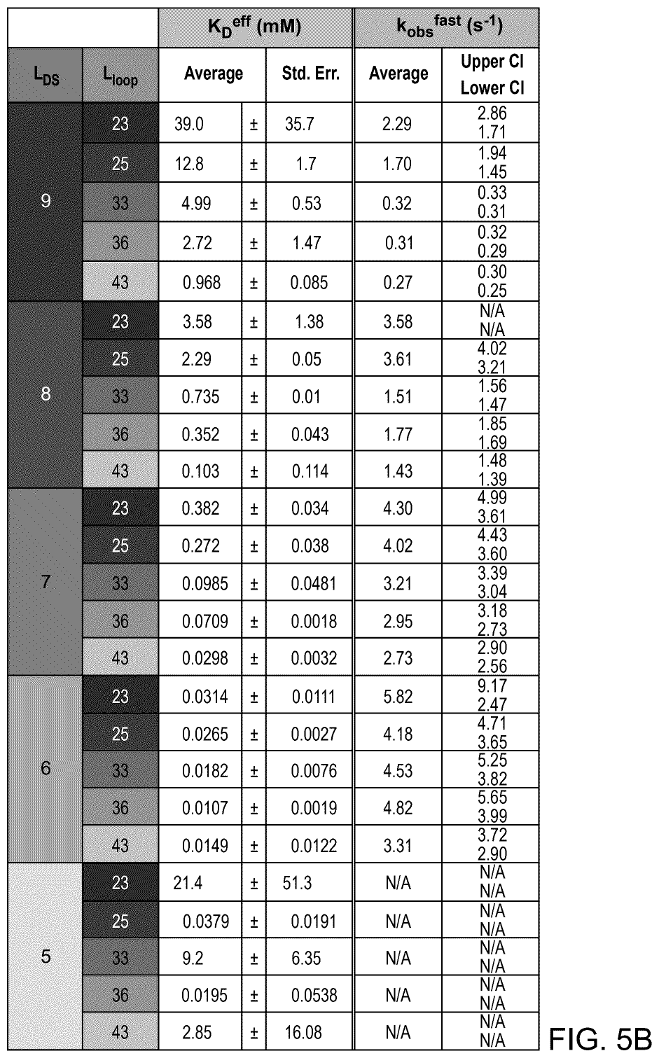

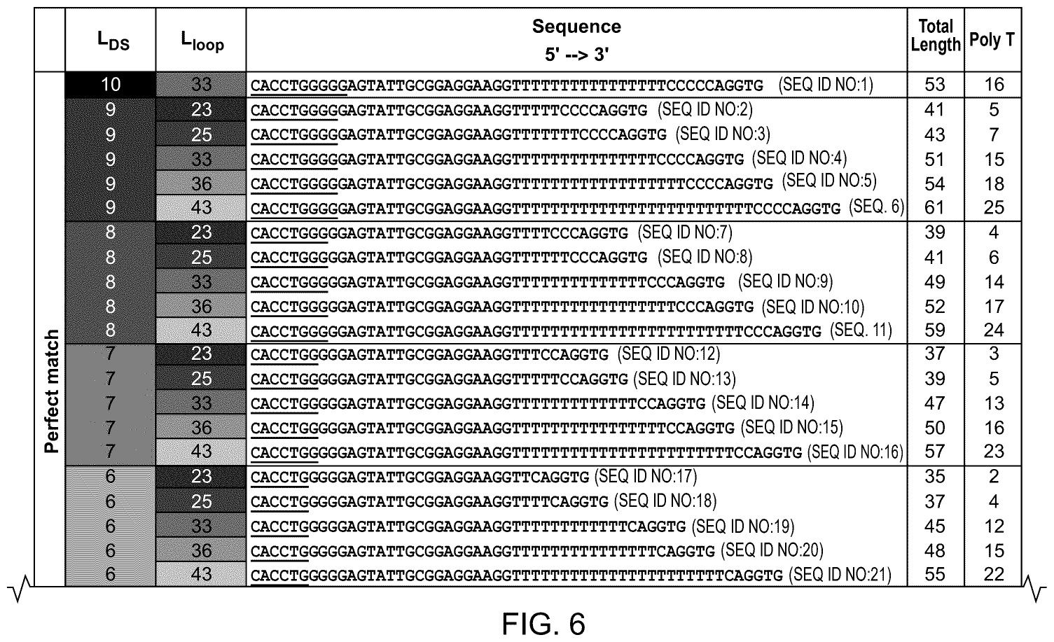

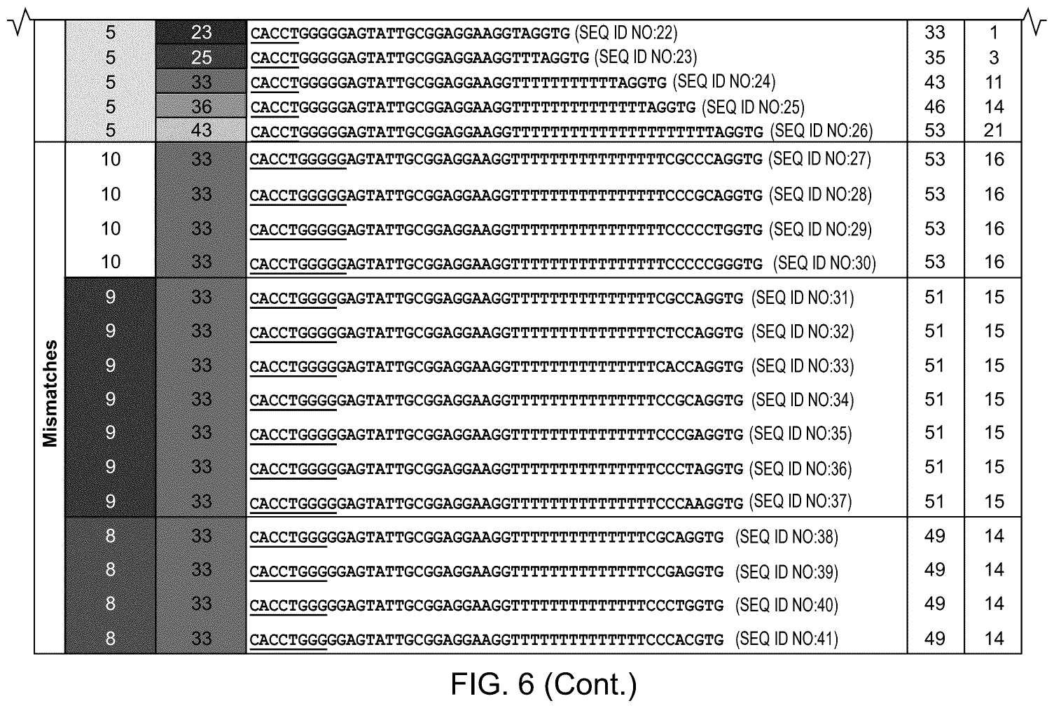

A model system was first used to mathematically test the anticipated effects (FIG. 4B) on binding response, and then these effects were confirmed with experimental results from an array of ISD switches (FIGS. 5A and 5B; FIG. 6) based on a well-studied ATP aptamer²⁰ with varying Lₗₒₒₚ and L_{DS}. All resulting constructs retain the high selectivity of the native aptamer (FIG. 3B). It was demonstrated conclusively that modulating Lₗₒₒₚ and L_{DS} in tandem decouples the control over the thermodynamics and kinetics of molecular recognition. Further, mismatches were introduced as a third tuning parameter to obtain even more precise enthalpic tuning and ultra-fast kinetics over a wide range of binding affinities.

### Example 2. Theoretical Principles of Molecular Switch Design

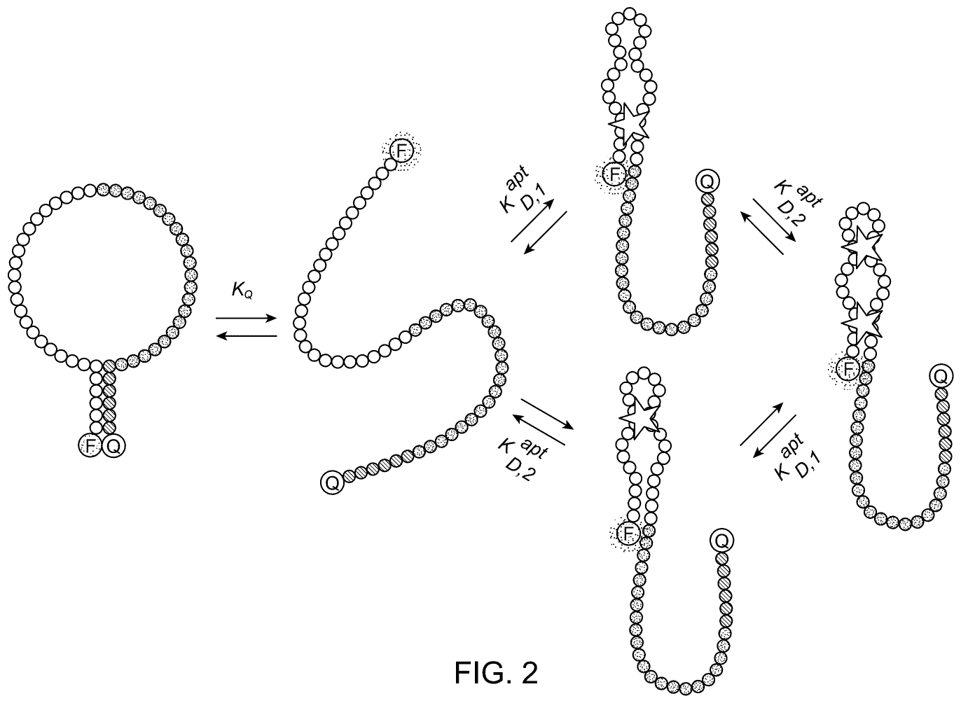

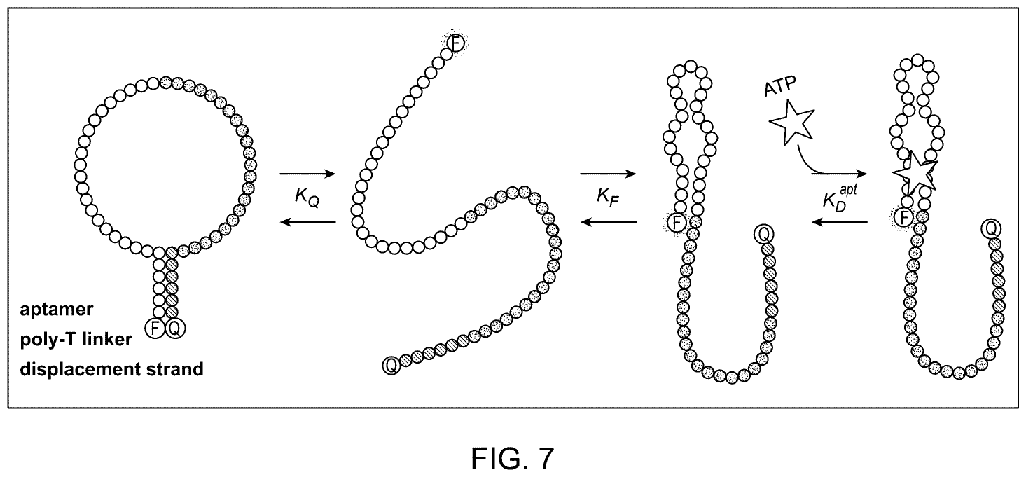

By examining a three-state population shift model, general insights were gained into how the design parameters affect the overall thermodynamics and kinetics of molecular recognition. An induced-fit model was used for its generalizability and simplicity, but derivations for conformational selection and two-site induced-fit binding were included (FIGS. 7 and 2, respectively). This inclusion is in recognition of the fact that the ATP aptamer used in this work has two binding sites²⁰ and can exhibit both induced-fit and conformational selection behavior^{21,22}.

It was assumed that the switch exists in an equilibrium between quenched (Q), unfolded (U), and target-bound (B) states (FIG. 1). The distributions of Q, U, and B are governed by the equilibrium constant (K_{Q}) for the intramolecular quenching reaction,

$${K}_{Q} = \frac{[ Q ]}{[ U ]} = \frac{{k}_{o n}^{DS}}{{k}_{off}^{DS}} \qquad ( 1 )$$

and the dissociation constant of the native aptamer

$$( {K}_{D}^{apt} ) ,$$

$${K}_{D}^{apt} = \frac{[ U ] [ T ]}{[ B ]} = \frac{{k}_{off}^{apt}}{{k}_{o n}^{apt}} . \qquad ( 2 )$$

Target binding to the aptamer depletes the unfolded state, shifting the quenching reaction towards the unfolded state, generating more signal. Assuming a quenching efficiency of η, this equilibrium shift generates a target concentration-dependent signal given by

$${S}_{eq} = {[ apt ]}_{total} \frac{1 + ( 1 - η ) {K}_{Q} + \frac{[ T ]}{{K}_{d}^{apt}}}{1 + {K}_{Q} + \frac{[ T ]}{{K}_{d}^{apt}}} . \qquad ( 3 )$$

The effective dissociation constant

$$( {K}_{d}^{eff} ) ,$$

which reflects the effective affinity of the overall equilibrium, can be derived as

$${K}_{d}^{eff} = {K}_{d}^{apt} ( 1 + {K}_{Q} ) . \qquad ( 4 )$$

Thus, while the

$${K}_{d}^{eff}$$

of the construct is defined by properties of the native aptamer

$$( {K}_{D}^{apt} ) ,$$

it also depends strongly on K_{Q}. Moreover, the background signal of the construct is also strongly related to K_{Q} by

$${S}_{background} = \frac{{[ apt ]}_{total}}{1 + {K}_{Q}} . \qquad ( 5 )$$

Therefore, it is crucial to understand the relative contributions of L_{DS} and Lₗₒₒₚ to K_{Q}. The effects of these independent tuning mechanisms are isolated by considering K_{Q} to be given by

$${K}_{Q} = \frac{{[ DS ]}_{eff}}{{K}_{d}^{DS}} , \qquad ( 6 )$$

where [DS]_{eff} is the effective concentration of the displacement strand that arises from covalent coupling to the native aptamer—a function of Lloop—and

$${K}_{d}^{DS}$$

represents the dissociation constant for the hybridization of an unlinked displacement strand—a function of LDS. [DS]_{eff} constitutes the entropic component of K_{Q}, whereas

$${K}_{d}^{DS}$$

constitutes the enthalpic component of K_{Q}. If the displacement strand is too short

$$( high {K}_{d}^{DS} )$$

or the linker is too long (low [DS]_{eff}), K_{Q} will be small, resulting in a large background (Eq. 5) and little signal change upon the addition of target (Eq. 3).

It was assumed that

$${K}_{D}^{DS}$$

is simply related to die binding energy of the free, untethered displacement strand, ΔG_{DS}:

$${K}_{D}^{DS} = exp ( \frac{Δ {G}_{DS}}{RT} ) , \qquad ( 7 )$$

where

$$Δ {G}_{DS} ≅ - 1.7 \frac{kcal}{mol bp} {L}_{DS} 23.$$

Arguments can also be made for the approximate scaling of K_{Q} with Lloop based on observations of rates of DNA hairpin closure as a function of loop size. Since the dissociation rate

$$( {k}_{off}^{DS} )$$

is relatively independent of Lloop and the association rate

$$( {k}_{on}^{DS} )$$

has been shown to scale inversely with Lₗₒₒₚ to the power of 2.6±0.3 (Ref. 24), it can be approximated that

$${K}_{Q} = \frac{{k}_{on}^{DS}}{{k}_{off}^{DS}}$$

scales as ˜Lloop-2.6. Therefore, decreases in LDS will be mirrored by an increase in KQ, shifting the equilibrium towards the quenched state, which results in decreased background signal at the expense of higher

$${K}_{D}^{eff} .$$

However, it can be derived that Lloop will have a subtler per-base effect on KQ relative to LDS and that increases in Lloop will have diminishing returns because

$$\frac{{dK}_{Q}}{{dL}_{loop}}$$

decreases as

$$\frac{1}{{L}_{loop}} .$$

This qualitative analysis also yields testable insights into the kinetic control of the system. It was discussed how Lₗₒₒₚ affects the relevant kinetic parameters, and others have shown that increasing hybridization strength decreases

$${k}_{off}^{DS}$$

much more than

$${k}_{o n}^{DS}$$

### (Ref. ²⁶ ).

Since the observed binding rate (k_{obs}) depends on the sum of

$${k}_{o n}^{DS} and {k}_{off}^{DS}$$

as

$${k}_{obs} = \frac{1}{2} ( {k}_{off}^{DS} + {k}_{o n}^{DS} + [ T ] {k}_{o n}^{apt} + {k}_{off}^{apt} + \sqrt{{( {k}_{off}^{DS} + {k}_{o n}^{DS} + [ T ] {k}_{o n}^{apt} + {k}_{off}^{apt} )}^{2} - 4 ( {k}_{o n}^{DS} {k}_{off}^{apt} + {k}_{off}^{DS} ( {k}_{off}^{apt} + [ T ] {k}_{o n}^{apt} ) )} ) , \qquad ( 8 )$$

it can be determined that decreasing Lₗₒₒₚ or L_{DS} will both increase k_{obs}. To summarize, it is expected that L_{DS} and Lₗₒₒₚ will have opposing effects on ISD switch thermodynamics but additive effects on the kinetics, and L_{DS} will have a more profound impact per base than Lₗₒₒₚ on effective binding affinity.

### Example 3. Experimental Characterization of the ISD Switch

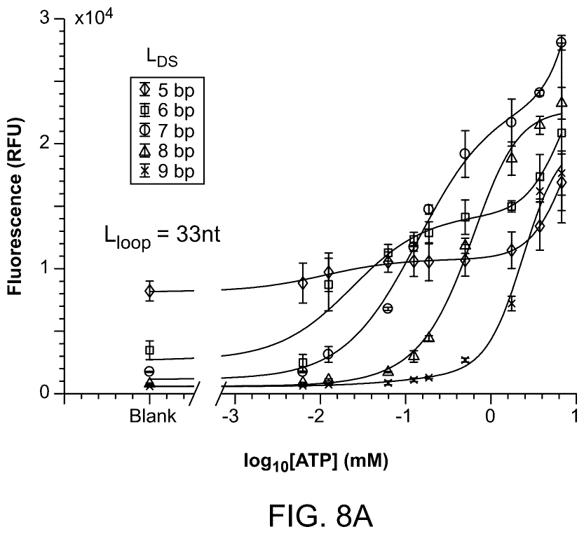

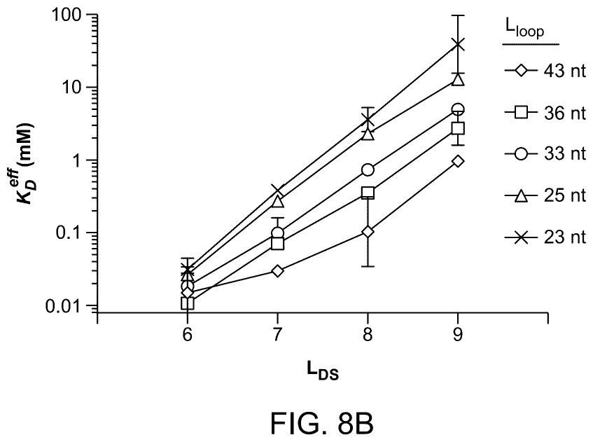

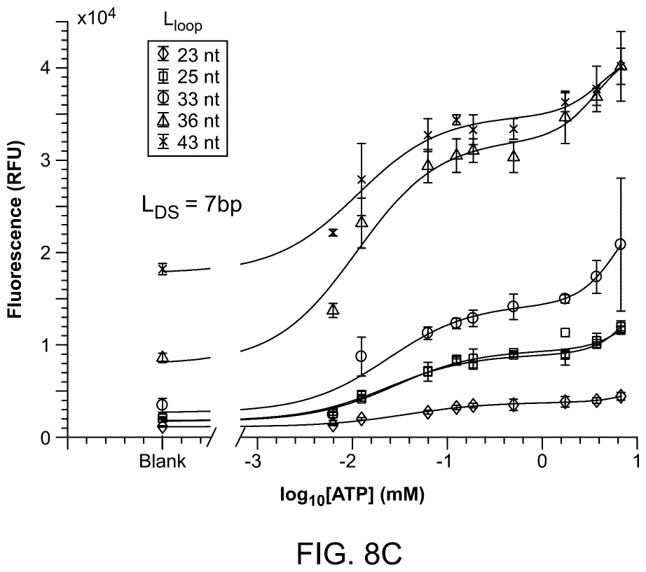

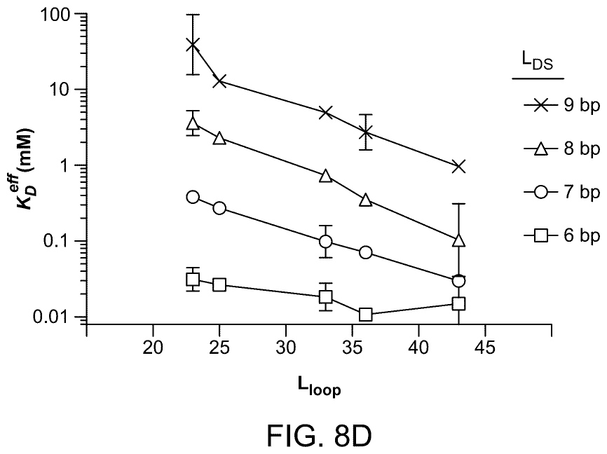

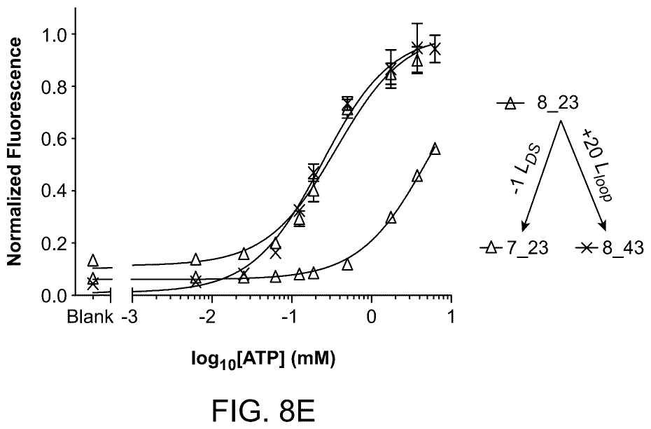

In order to experimentally validate the predictions of this model, ligand binding was characterized for over 25 ISD switches (FIGS. 5A and 5B) derived from the same ATP aptamer²⁰. Displacement strands with L_{DS} ranging from 5-9 base pairs (bp) and poly-T linkers of various lengths to yield Lₗₒₒₚ ranging from 23-43 nucleotides (nt) were introduced. As expected, increasing L_{DS} with a constant Lₗₒₒₚ resulted in decreased background signal and lower apparent affinity (FIG. 8A). Fits equation S2 reveal a clear trend in which

$${K}_{d}^{eff}$$

increases with L_{DS}, reflecting a reduction in effective binding affinity (FIG. 8B). It was observed that

$${K}_{d}^{eff}$$

can be increased by up to 1,200-fold by the removal of three bases from the displacement strand (e.g., 6_23 to 9_23), with an average fold increase of ˜6.7±2.4 per base. Notably, the addition or removal of a single base from the displacement strand can shift

$${K}_{D}^{eff}$$

by more than an order of magnitude.

$${K}_{D}^{eff} ,$$

In contrast, it was observed that changing Lₗₒₒₚ has a subtler per-base effect on with just a ˜0.83±0.15 fold decrease in

$${K}_{D}^{eff}$$

per additional base (FIGS. 8C and 8D). On average, the linker must be decreased by 17.7±11.9 nt in order to shift the binding curve by an amount equivalent to the addition of a single base to the displacement strand. This loop/base equivalence value varies from construct to construct but is epitomized by the observation that adding 20 nt to the linker of 8_23 (generating 8_43) results in the same

$${K}_{D}^{eff}$$

as removing 1 bp from the displacement strand of 8_23 (generating 7_23) (FIG. 8E). The vast difference in these effects enabled the modulation of effective binding affinity both finely (by tuning Lₗₒₒₚ) and over a wide functional range (by tuning L_{DS}).

For equation S2: The bimodal K_{D} behavior was recovered by allowing site 1 and site 2 to vary in fluorescence values. Without this assumption, the model in FIG. 2 would result in a binding curve with a concave second derivative. It was assumed that binding to site 1 and binding to site 2 have different fluorescence values, η₁ and η₂. K_{D,1} and K_{Q} were extracted via:

$$Signal = {B}_{max} \frac{{K}_{D , 1} {K}_{D , 2} + {η}_{1} {K}_{D , 1} [ T ] + {η}_{2} {K}_{D , 2} [ T ] + {K}_{D , 1} {{K}_{D , 2} [ T ]}^{2}}{{K}_{D , 1} {K}_{D , 2} ( 1 + {K}_{Q} ) + {K}_{D , 1} [ T ] + {K}_{D , 2} [ T ] + {K}_{D , 1} {{K}_{D , 2} [ T ]}^{2}} \qquad ( S2 )$$

$${K}_{D}^{eff} was reported as calculated by equation 4 , using {K}_{D , 1} .$$

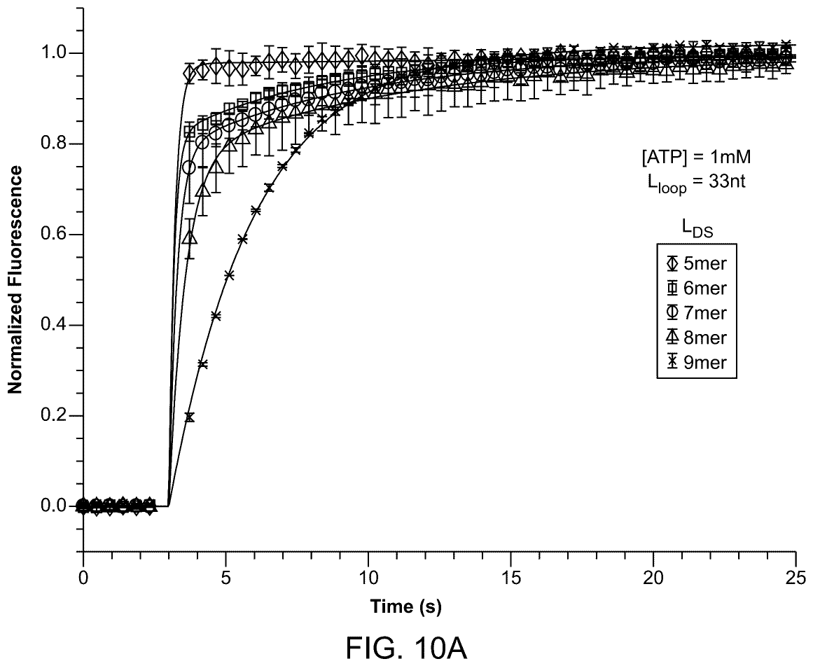

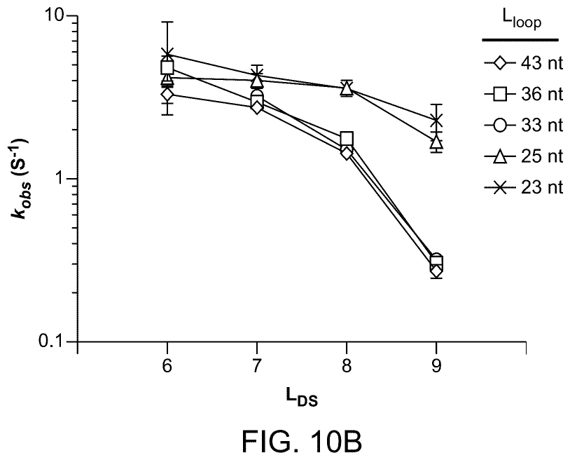

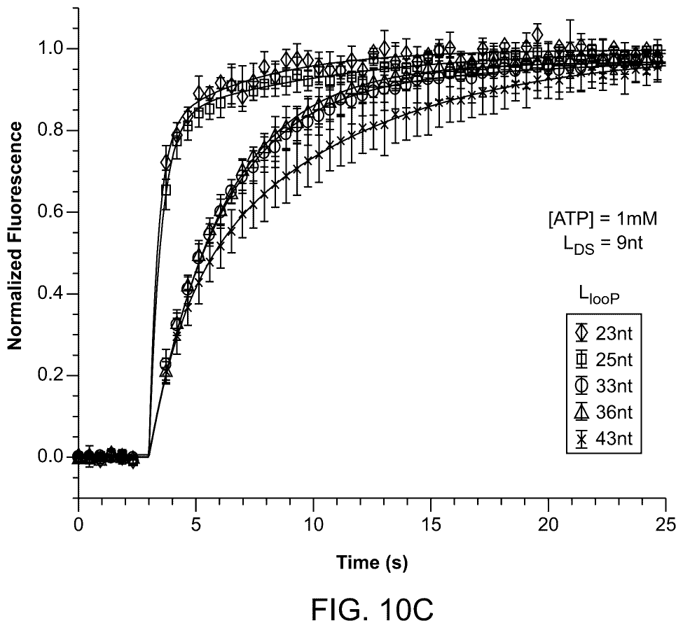

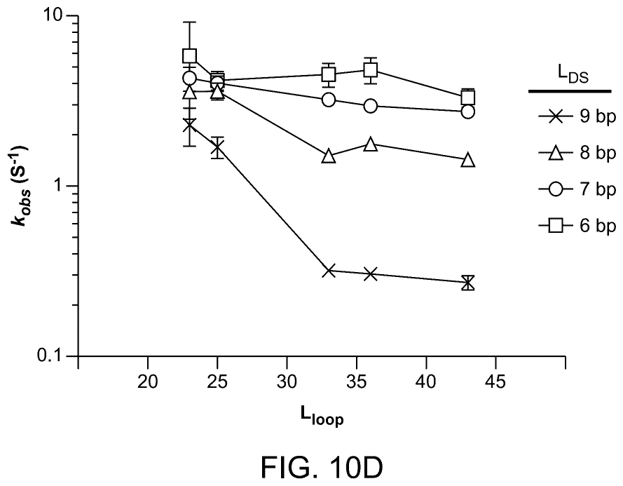

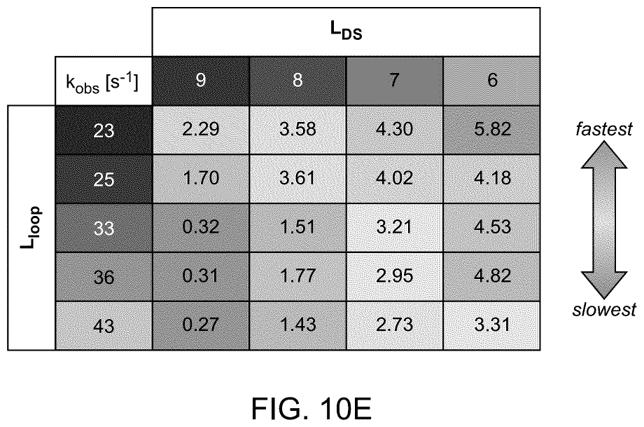

Next, the temporal response of molecular recognition for all of the constructs was measured to validate the previously described kinetic contributions of Lₗₒₒₚ and L_{DS}. As anticipated, it was found that decreasing L_{DS} with a constant Lₗₒₒₚ (FIGS. 10A and 10B) or decreasing Lₗₒₒₚ with a constant L_{DS} (FIGS. 10C and 10D) results in faster temporal responses. By combining these two tuning mechanisms, one can vary the switch time constant (k_{obs}⁻¹) by over 20-fold, ranging from ˜3 seconds to ˜170 milliseconds. It is noted that even the slowest constructs represent a marked improvement over traditional aptamer beacons, which typically exhibit time constants on the order of minutes to hours ^{16,27}. The fast kinetics of the ISD switch are attributable to a much higher

$${k}_{o n}^{DS}$$

resulting from the large effective concentration of the displacement strand. This high

$${k}_{o n}^{DS}$$

allows the use of much shorter displacement strands than are possible with aptamer beacons, which in turn results in much faster

$${k}_{off}^{DS} .$$

The simultaneous increase in both

$${k}_{o n}^{DS} and {k}_{off}^{DS}$$

greatly increases k_{obs} (Eq. 8). Indeed, switches with L_{DS}=5 achieved temporal resolution exceeding the time resolution of the detector—with a time delay of 465 ms between injection and measurement,

$${k}_{obs}^{MAX}$$

is approximately 10 s⁻¹.

### Example 4. Decoupling Thermodynamics and Kinetics

The thermodynamic and kinetic findings introduce the possibility of designing ISD switches in which temporal resolution can be tuned completely independently of binding affinity. Since the effective affinity of the construct depends on the equilibrium constant for hairpin formation,

$${K}_{Q} = \frac{{k}_{on}^{DS}}{{k}_{off}^{DS}} ,$$

it is clear that the binding kinetics can be increased while maintaining the same effective affinity as long as the ratio between association and dissociation rates is preserved. Decreasing Lₗₒₒₚ increases the binding rate and decreases the effective affinity of the construct. This decrease in affinity can be offset by shortening L_{DS}, which in turn results in a net additive increase in temporal resolution. Based on the observed dependencies of the two control parameters, it was hypothesized that it should be feasible to achieve faster switching responses and maintain effective affinity by decreasing Lloop and LDS simultaneously.

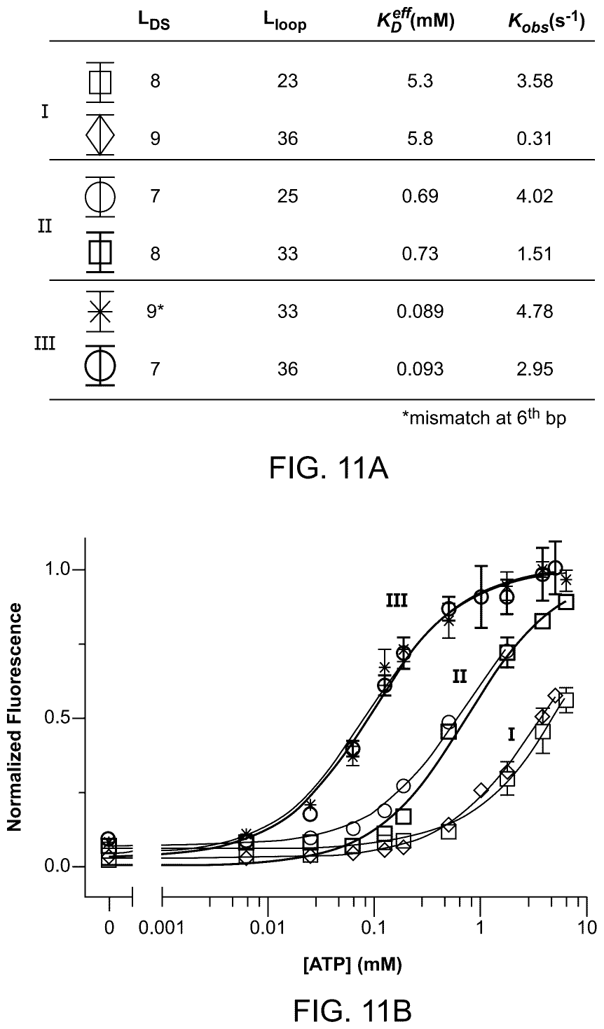

This prediction was confirmed with three pairs of constructs (FIG. 11A) that each have statistically indistinguishable effective affinities (FIG. 11B). Constructs with longer L_{DS} and Lₗₒₒₚ had universally slower temporal resolution (FIG. 11C). Tandem tuning of the two parameters allowed the increase of the aptamer temporal response by up to 6-fold without changing

$${K}_{D}^{eff} .$$

Importantly, this tunability was accomplished over a wide range of effective affinities (˜90 μM to ˜5.8 mM).

### Example 5. Precision Tuning Through Displacement Strand Mismatches

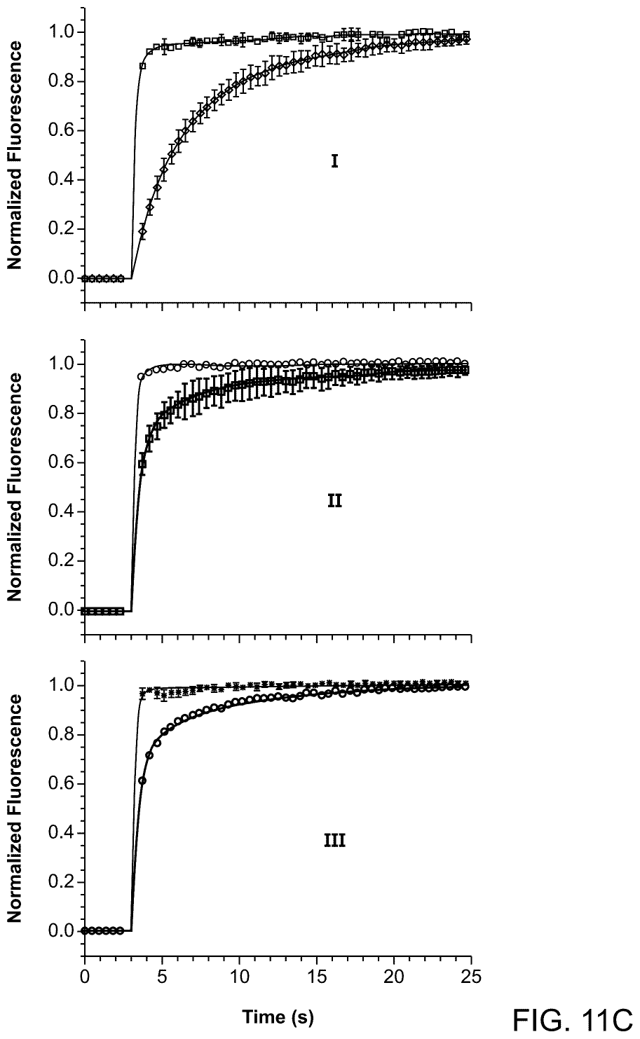

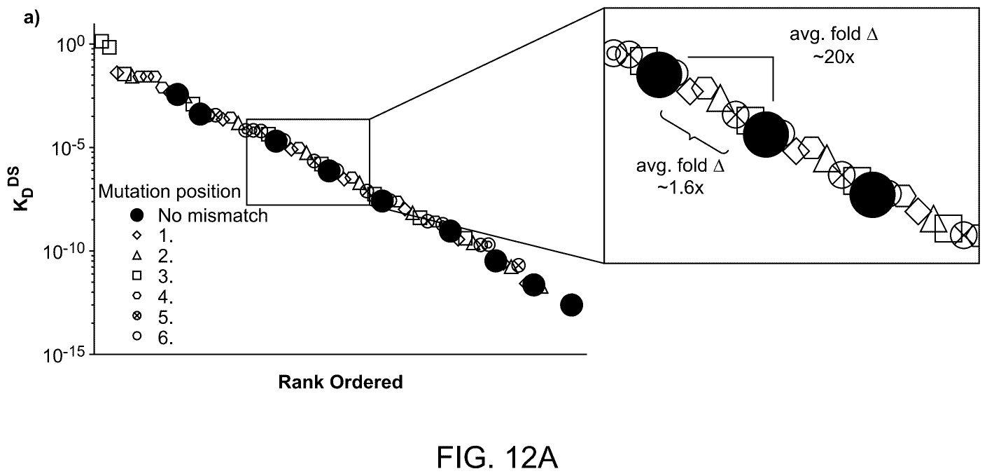

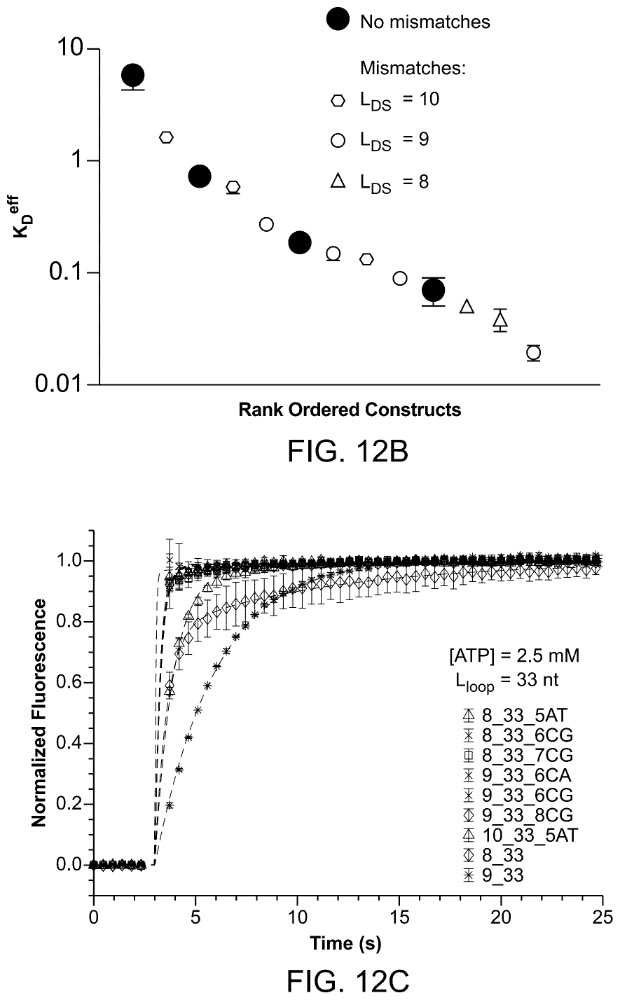

It was hypothesized that even finer control over the hybridization strength of the duplexed region of the ISD construct should be possible if the complementarity of the two strands was manipulated. Indeed, by expanding the design space to include single-base mismatches, it was calculated that the theoretical resolution of the tuning could increase more than 10-fold relative to that of only perfectly-matched displacement strands (FIG. 12A). Since the introduction of mismatches has been shown to drastically increase k_{off} and kₒₙ for two hybridizing strands, it was anticipated that the introduction of mismatches would also greatly increase the observed kinetics²⁶. Thus, mismatches should enable finer enthalpic control over the binding curve and enhance the ability to increase kinetics independently of affinity.

To experimentally confirm these predictions, single mismatches of different identities (A, G, C or T) were introduced at various positions throughout the displacement strands of three constructs: 8_33 (L_{DS}=8, Lₗₒₒₚ=33), 9_33 (L_{DS}=9, Lₗₒₒₚ=33), and 10_33 (L_{DS}=10, Lₗₒₒₚ₌₃₃). Upon comparing the thermodynamic properties of the original constructs to those containing the mismatches, it was found that one was able to obtain more closely spaced binding curves based on the position and identity of the mismatch. For constructs with a 33-nt loop, for example, the average distance in

$${K}_{D}^{eff}$$

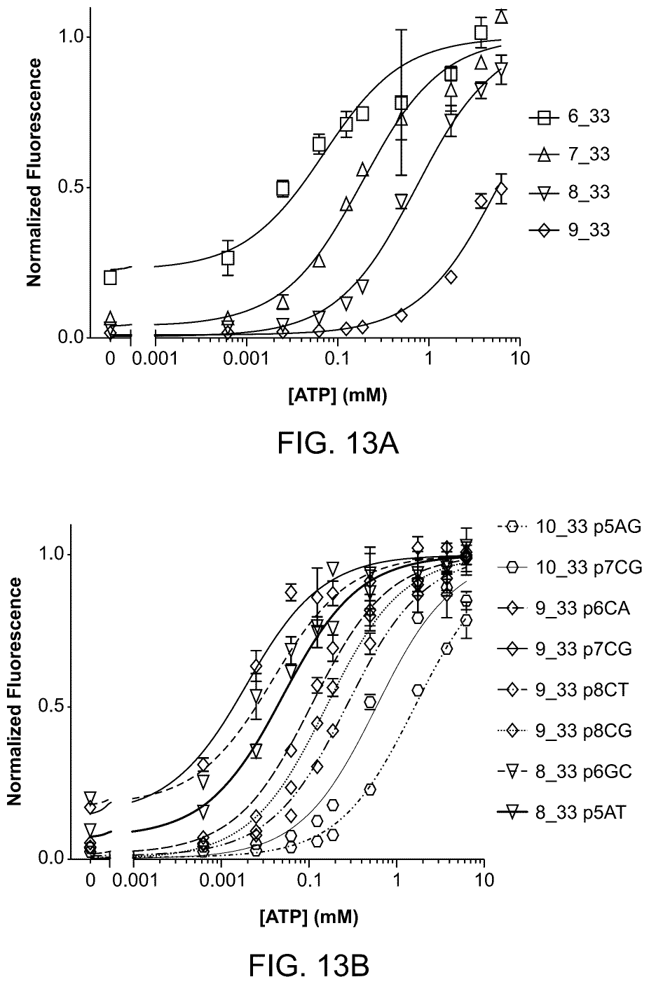

values that could be obtained by modulating L_{DS} was 6.55±1.01 (FIG. 13A). However, using just a small subset of possible mismatches, one was able to achieve increments of 1.72±0.34 (FIG. 13B; FIG. 12B). Therefore, by modulating the position and identity of mismatches, one can generate sets of constructs that yield much finer enthalpic control than would be possible by changing L_{DS} alone. Lastly, the incorporation of mismatches substantially increases

$${k}_{off}^{DS}$$

(Ref. ²⁶), such that mismatches not only confer greater control over the thermodynamics but also dramatically increase temporal resolution relative to perfectly-matched displacement strands (FIG. 12C).

Aptamer-based molecular switches are powerful tools in biotechnology, but their utility has been constrained by a limited ability to rationally engineer their binding characteristics in terms of target affinity and kinetics. Prior studies have indicated that the thermodynamics and kinetics of such switches are coupled in such a manner that gains in one parameter will generally result in sacrifices in the other¹⁶. The present disclosure demonstrates that an ISD aptamer switch construct that allows remarkably precise independent control of both thermodynamic and kinetic parameters. The construct couples an existing aptamer with a partially complementary displacement strand via a poly-T linker, such that alterations in the length of either feature can meaningfully shift the ISD equilibrium. A mathematical model was used to demonstrate how changes in L_{DS} would be predicted to confer coarse control over ISD switch affinity; at the same time, changes in Lₗₒₒₚ should produce a subtler effect per base added or removed. This expectation was subsequently confirmed experimentally and the capacity to carefully manipulate the binding characteristics of the switch through these two parameters was demonstrated. For example, binding kinetics can be increased by an order of magnitude with minimal effect on aptamer affinity by selectively shortening both L_{DS} and Lₗₒₒₚ. Even finer tuning of ISD properties is possible with further manipulation of the strength of displacement strand hybridization through the targeted introduction of individual base-mismatches into the displacement strand sequence.

As the desire for rapid molecular detection becomes prevalent, so will the need to tune the kinetics of molecular recognition independently of binding affinity. The present approach is advantageous in this regard, as it offers opportunities for control that exceed those of existing molecular switch designs, which are generally constrained by tight coupling of kinetic and thermodynamic parameters and offer less freedom for structural manipulation. For example, the feasibility of achieving ultrafast kinetic responses (on the order of hundreds of milliseconds) was demonstrated with the ISD constructs without meaningfully sacrificing target affinity, whereas aptamer beacons typically exhibit kinetics on the order of minutes to hours. Critically, the present molecular switch design should be compatible with virtually any aptamer sequence, making it feasible to design optimized molecular switches that are ideally suited for a diverse array of biotechnology and synthetic biology applications.

### Example 6. General Methods

Reagents. All chemicals were purchased from Thermo Fisher Scientific unless otherwise noted, including ATP (25 μmol, 100 mM), Tris-HCl Buffer (1 M, pH 7.5), magnesium chloride (1 M, 0.2 μm filtered), and Hyclone molecular biology-grade water (nuclease-free). Oligonucleotides modified with Cy3 fluorophore at the 5′ ends and DABCYL quencher at the 3′ ends, both purified by HPLC, were purchased from Integrated DNA Technologies. All sequences used in this work are shown in FIG. 6. All oligonucleotides were resuspended in nuclease-free water and stored at −20° C.

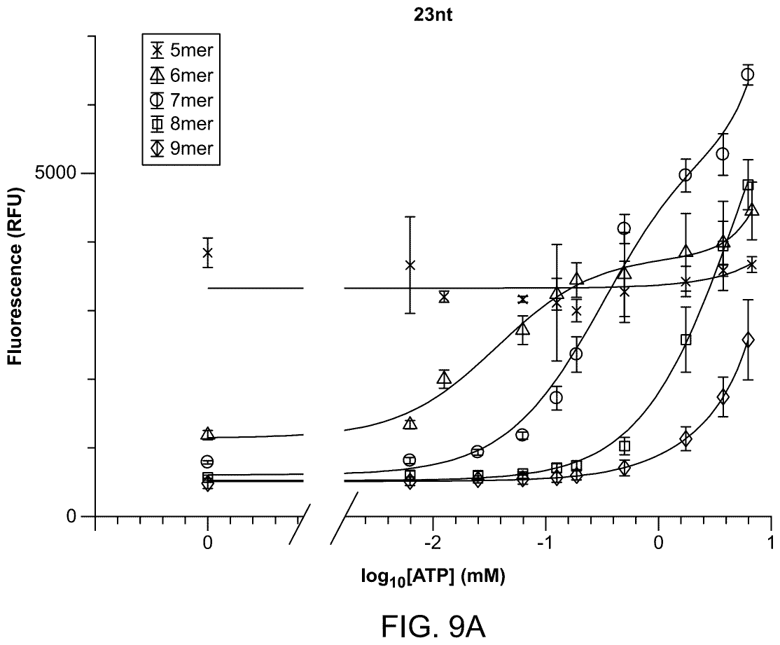

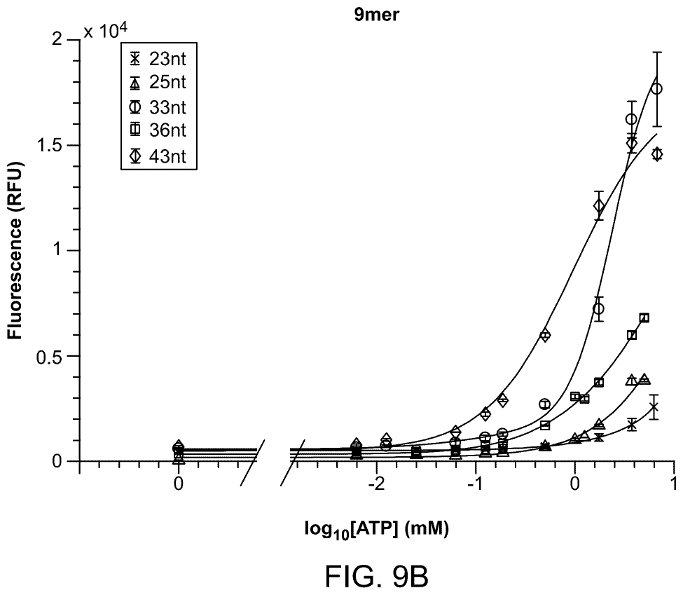

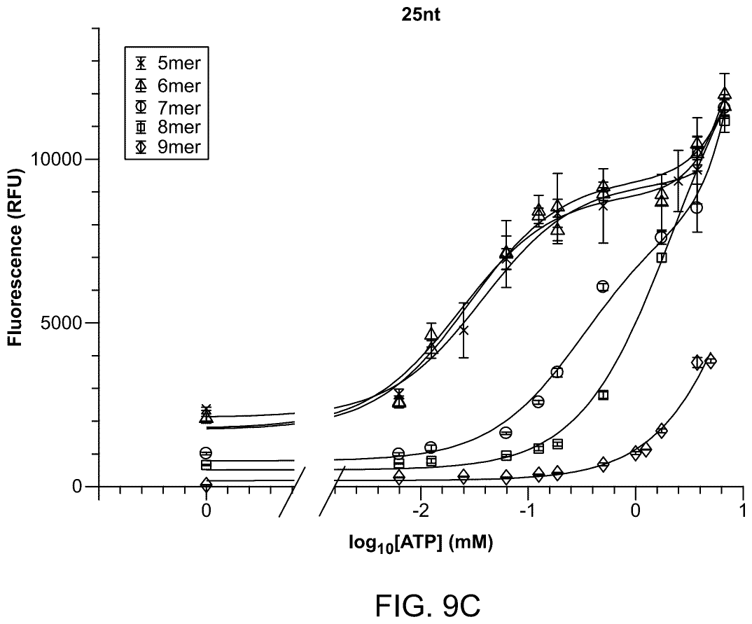

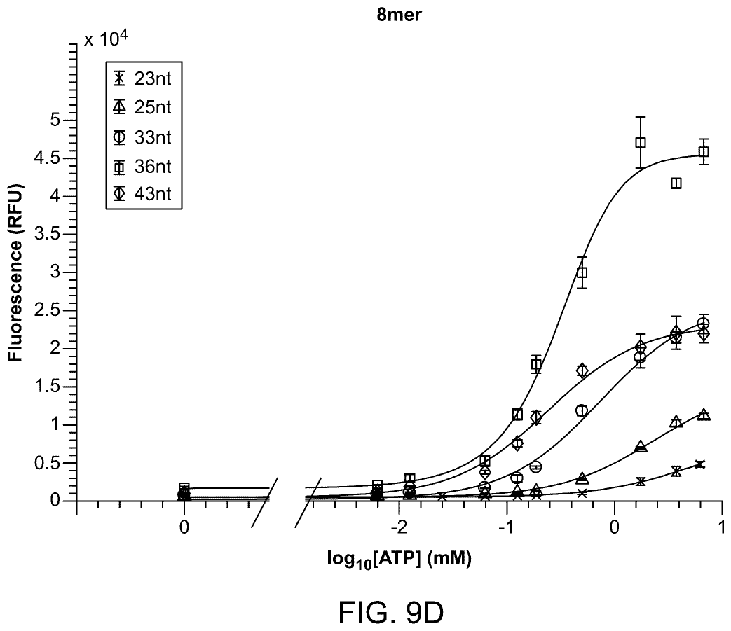

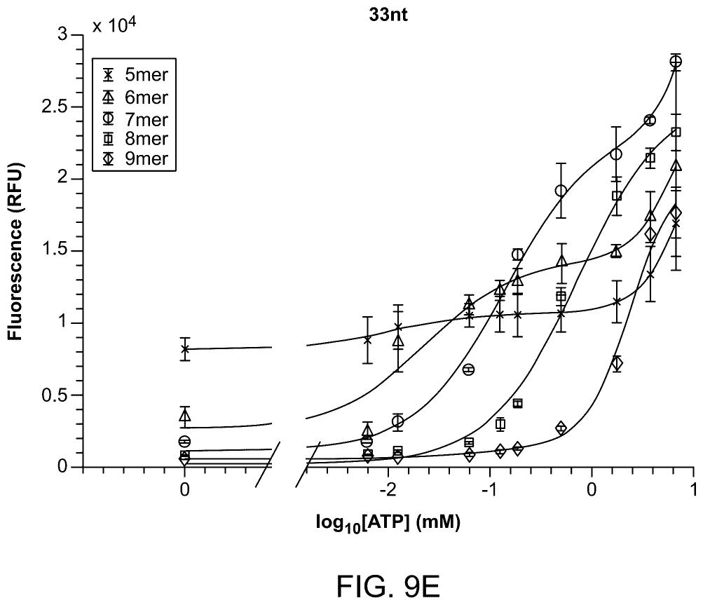

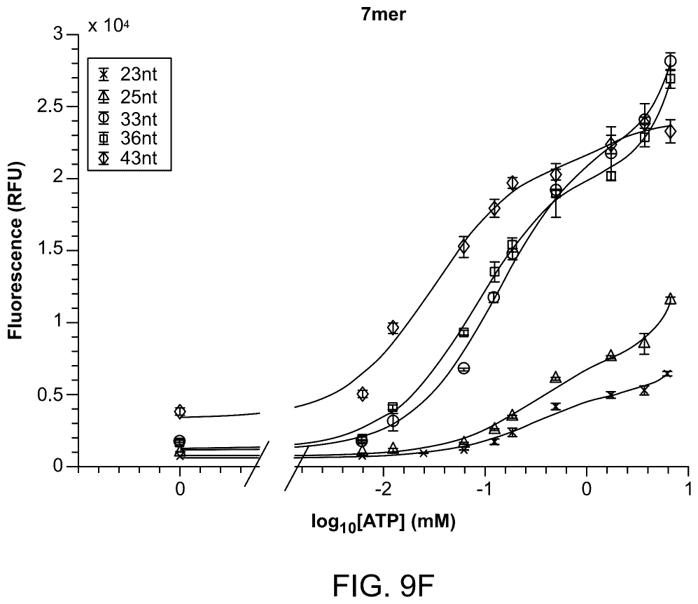

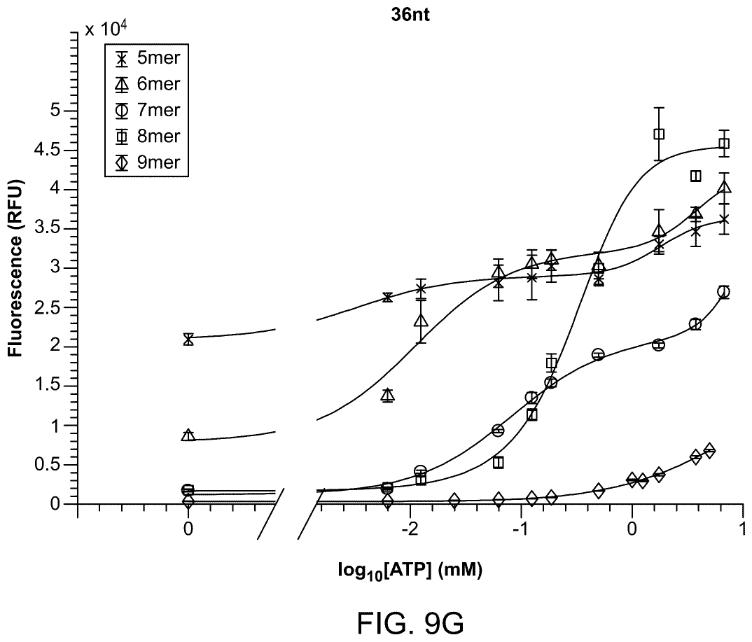

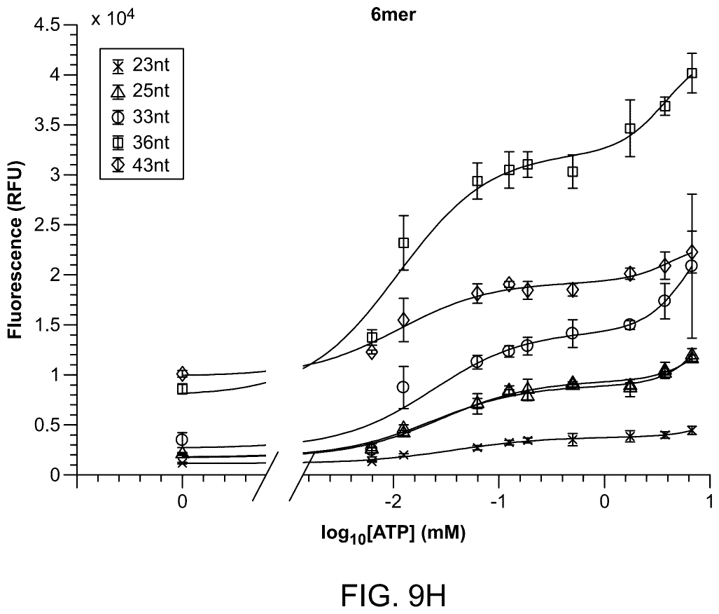

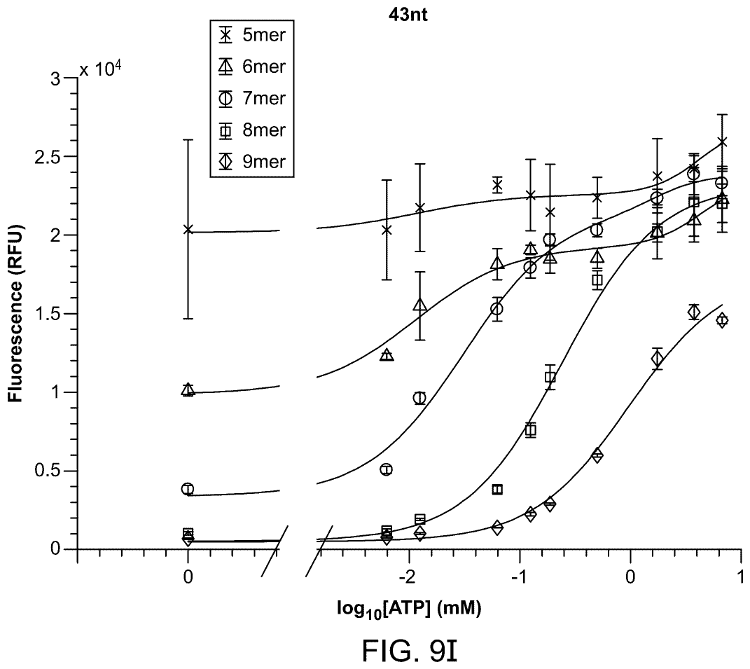

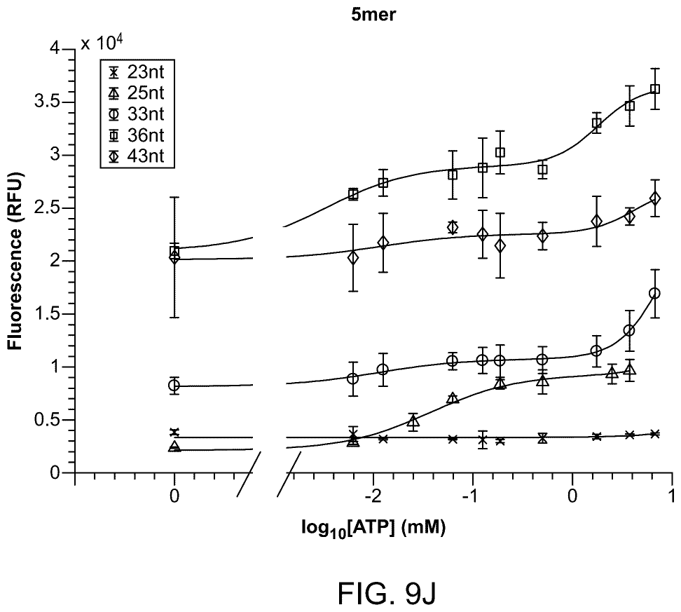

Measurements of Effective Binding Affinity. To obtain binding curves, 40 μL reactions were prepared in 1× ATP binding buffer (10 mM Tris-HCl, pH 7.5 and 6 mM MgCl₂) with 250 nM aptamer and final ATP concentrations in the range of 6.25 μM to 6.75 mM. The fluorescence spectra for all samples were measured at 25° C. on a Synergy H1 microplate reader (BioTeK). Emission spectra were monitored in the 550-700 nm range with Cy3 excitation at 530 nm and a gain of 100, in 96-well plates. All measurements were performed in triplicate. Representative concentration-dependent emission spectra are shown in FIGS. 9A-9J.

Measurements of Binding Kinetics. ISD constructs of varying linker and displacement strand lengths were suspended at a concentration of 333.3 nM in a 30 μL total volume of 1× ATP binding buffer (10 mM Tris-HCl, pH 7.5 and 6 mM MgCl₂). Kinetic fluorescence measurements of the quencher-fluorophore pair were made using a Synergy H1 microplate reader. Cy3 was excited at 530 nm, and unquenched fluorescence was measured at 570 nm emission using monochromators at the minimum possible regular time interval of 0.465 seconds. After timed injection of 10 μL ATP (final [ATP]=0, 1, or 2.5 mM) in 1× ATP binding buffer into the 30 μL ISD solution, the kinetic response was measured in the 40 μL sample volume. All kinetic data was first normalized relative to the 0 μM target concentration to account for the effect of sample volume change upon injection with ATP. For plotting, the curves were normalized to range from 0 to 1 in order to visually emphasize changes in rate constants rather than the background and peak levels that are dictated by the thermodynamics. All measurements were performed in triplicate.

Thermodynamic Analysis. Three replicates of inputs (X=log ([ATP]) in mM) versus outputs (Y in raw RFU intensity) were fit individually for each construct to extract the effective binding affinity. The resultant parameters from fitting X and Y to equation S2 were averaged over the three independent fits. The logarithmic values of thermodynamic constants pK_{D1}=−log (K_{D1}), pK_{D2}=−log (K_{D2}), and p_{KQ}=−log (K_{Q}) were fit such that equation S2 becomes:

$$Y = {B}_{max} \frac{\begin{matrix}{10}^{{pK}_{D 1}} {10}^{{pK}_{D 2}} + {η}_{1} {10}^{{pK}_{D 1}} {10}^{X} + \\ {η}_{2} {10}^{{pK}_{D 2}} {10}^{X} + {10}^{{pK}_{D 1}} {10}^{{pK}_{D 2}} {10}^{2 X}\end{matrix}}{\begin{matrix}{10}^{{pK}_{D 1}} {10}^{{pK}_{D 2}} ( 1 + {K}_{Q} ) + {10}^{{pK}_{D 1}} {10}^{X} + \\ {10}^{{pK}_{D 2}} {10}^{X} + {10}^{{pK}_{D 1}} {10}^{{pK}_{D 2}} {10}^{2 X}\end{matrix}} \qquad ( 9 )$$

Fits were performed via MATLAB's lsqcurve function with initial guesses equal to

$${B}_{max}^{guess} = max ( Y ) - min ( Y ) , {η}_{1}^{guess} = 1 , \\ {pK}_{D 1}^{guess} = - X ( Y ∼ \frac{max ( Y ) + min ( Y )}{2} ) , {η}_{2}^{guess} = 1 ,$$

$${pK}_{D 2}^{guess} = {pK}_{D 1}^{guess} - 6 , and {pK}_{Q}^{guess} = 1 - \frac{max ( Y ) - min ( Y )}{2 min ( Y )} .$$

Upper bounds were set to

$${B}_{max}^{upper} = max ( Y ) * 10 , {η}_{1}^{upper} = 1.01 , {pK}_{D 1}^{upper} = 1 , \\ {η}_{2}^{upper} = 1.01 , {pK}_{D 2}^{upper} = 10 , and {pK}_{Q}^{upper} = 10.$$

Lower bounds were set to

$${B}_{max}^{lower} = \frac{max ( Y )}{2} , {η}_{1}^{lower} = 0 , {pK}_{D 1}^{lower} = - 6 , {η}_{2}^{lower} = 0 ,$$

$${pK}_{D 2}^{lower} = - 3 , and {pK}_{Q}^{lower} = - 10.$$

Fitting was performed using a maximum of 100,000 iterations. pK_{D1} and pK_{Q} were averaged over at least three replicate fits for each construct. The effective binding affinity,

$${K}_{D}^{eff} ,$$

was then calculated by

$${K}_{D}^{eff} = {10}^{- {pK}_{D 1}} ( 1 + {10}^{- {pK}_{Q}} ) , \qquad ( 10 )$$

with the standard error given by propagation of errors

$${σ}_{{K}_{D}^{eff}} = \sqrt{\begin{matrix}{( ln ( 10 ) {σ}_{{pK}_{D 1}} {10}^{- {pK}_{D 1}} ( 1 + {10}^{- {pK}_{Q}} ) )}^{2} + \\ {( ln ( 10 ) {σ}_{{pK}_{Q}} {10}^{- {pK}_{D 1}} {10}^{- {pK}_{Q}} )}^{2}\end{matrix}} . \qquad ( 11 )$$

The curves plotted in FIGS. 8A and 8C were fit to the average of the three replicates.

Kinetic Analysis. Kinetic data were first normalized to a zero ATP control to account for changes in volume due to the injection of ATP. Each replicate was fit individually to

$$y ( t ) = \{ \begin{matrix}A - B exp ( - {k}_{1} t ) - C exp ( - {k}_{2} t ) & t > {t}_{0} \\ A - B - C & t \leq {t}_{0}\end{matrix} \} , \qquad ( 12 )$$

$$where {k}_{1} > {k}_{2} .$$

Each replicate was then normalized to range from zero to one by:

$${y}^{*} ( t ) = \frac{y - A + B + C}{N} \qquad ( 13 )$$

where

$$N = \{ \begin{matrix}B & {k}_{2} \leq 0.015 {s}^{- 1} \\ B + C & {k}_{2} > 0.015 {s}^{- 1}\end{matrix} \} .$$

A piecewise function was used for N to control for an artifact of the fitting function, wherein if there is no observable k₂, the fit function still forces the fit to k₂ which can result in extremely large values of C. Therefore, C was omitted from the normalization if k₂ is very slow. The normalized responses were averaged together and the average response was again fit to equation 12 to obtain a rate representative of all three trials. Rate constants are reported as the best fit values ±95% upper/lower confidence intervals.

### REFERENCES

1. Motlagh, H. N., Wrabl, J. O., Li, J. & Hilser, V. J. The ensemble nature of allostery. Nature 508, 331-339 (2014).
2. Mandal, M. & Breaker, R. R. Gene regulation by riboswitches. Nat. Rev. Mol. Cell Biol. 5, 451-463 (2004).
3. Autour, A. et al. Fluorogenic RNA Mango aptamers for imaging small non-coding RNAs in mammalian cells. Nat. Commun. 9, (2018).
4. Bates, M., Huang, B., Dempsey, G. T. & Zhuang, X. Multicolor super-resolution imaging with photo-switchable fluorescent probes. Science 317, 1749-53 (2007).
5. Ferguson, B. S. et al. Real-Time, Aptamer-Based Tracking of Circulating Therapeutic Agents in Living Animals. Sci. Transl. Med. 5, 213ra165-213ra165 (2013).
6. Hernandez, F. J., Hernandez, L. I., Pinto, A., Schäfer, T. & Özalp, V. C. Targeting cancer cells with controlled release nanocapsules based on a single aptamer. Chem. Commun. 49, 1285-1287 (2013).
7. Wang, J. et al. Assembly of aptamer switch probes and photosensitizer on gold nanorods for targeted photothermal and photodynamic cancer therapy. ACS Nano 6, 5070-5077 (2012).
8. Harroun, S. G. et al. Programmable DNA switches and their applications. Nanoscale 10, 4607-4641 (2018).
9. Nutiu, R. & Li, Y. In vitro selection of structure-switching signaling aptamers. Angew. Chemie—Int. Ed. 44, 1061-1065 (2005).
10. Oh, S. S., Plakos, K., Lou, X., Xiao, Y. & Soh, H. T. In vitro selection of structure-switching, self-reporting aptamers. Proc. Natl. Acad. Sci. U. S. A. 107, 14053-14058 (2010).
11. Hamaguchi, N., Ellington, A. & Stanton, M. Aptamer beacons for the direct detection of proteins. Analytical Biochemistry 294, 126-131 (2001).
12. Munzar, J. D., Ng, A. & Juncker, D. Duplexed aptamers: History, design, theory, and application to biosensing. Chem. Soc. Rev. 48, 1390-1419 (2019).
13. Feagin, T. A., Maganzini, N. & Soh, H. T. Strategies for Creating Structure-Switching Aptamers. ACS Sensors 3, 1611-1615 (2018).
14. Vallee-Belisle, A. & Francesco Ricci, and K. W. Plaxco. Thermodynamic basis for the optimization of binding-induced biomolecular switches and structure-switching biosensors. Proc. Natl. Acad. Sci. 106, 13802-13807 (2009).
15. Mariottini, D., Idili, A., Nijenhuis, M. A. D., De Greef, T. F. A. & Ricci, F. DNA-Based Nanodevices Controlled by Purely Entropic Linker Domains. Journal of the American Chemical Society 140, 14725-14734 (2018).
16. Hall, B., Cater, S., Levy, M. & Ellington, A. D. Kinetic optimization of a protein-responsive aptamer beacon. Biotechnol. Bioeng. 103, 1049-1059 (2009).
17. Nutiu, R. & Li, Y. Structure-Switching Signaling Aptamers: Transducing Molecular Recognition into Fluorescence Signaling. Chem.-A Eur. J. 10, 1868-1876 (2004).
18. Tang, Z. et al. Aptamer Switch Probe Based on Intramolecular Displacement. J. Am. Chem. Soc. 130, 11268-11269 (2008).
19. Huizenga, D. E. & Szostak, J. W. A DNA Aptamer That Binds Adenosine and ATP. Biochemistry 34, 656-665 (1995).
20. Zhang, Z., Oni, O. & Liu, J. New insights into a classic aptamer: Binding sites, cooperativity and more sensitive adenosine detection. Nucleic Acids Res. 45, 7593-7601 (2017).
21. Munzar, J. D., Ng, A. & Juncker, D. Comprehensive profiling of the ligand binding landscapes of duplexed aptamer families reveals widespread induced fit. Nat. Commun. 9, (2018).
22. Xia, T., Yuan, J. & Fang, X. Conformational dynamics of an ATP-binding DNA aptamer: A single-molecule study. J. Phys. Chem. B 117, 14994-15003 (2013).
23. SantaLucia, J. A unified view of polymer, dumbbell, and oligonucleotide DNA nearest-neighbor thermodynamics. Proc. Natl. Acad. Sci. 95, 1460-5 (1998).
24. Bonnet, G., Krichevsky, O. & Libchaber, A. Kinetics of conformational fluctuations in DNA hairpin-loops. Biophysics (Oxf)95, 8602-8606 (1998).
25. Wang, X. & Nau, W. M. Kinetics of End-to-End Collision in Short Single-Stranded Nucleic Acids. J. Am. Chem. Soc. 126, 808-813 (2004).
26. Xu, S. et al. Real-time reliable determination of binding kinetics of DNA hybridization using a multi-channel graphene biosensor. Nat. Commun. 8, 1-10 (2017).
27. Nutiu, R. & Li, Y. Structure-switching signaling aptamers. J. Am. Chem. Soc. 125, 4771-4778 (2003).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An aptamer switch polynucleotide comprising:
an aptamer, wherein the aptamer is linked to a first label at a first terminus of the aptamer;
a polynucleotide linker, wherein a first terminus of the polynucleotide linker is linked to a second terminus of the aptamer; and
a displacement strand, wherein a first terminus of the displacement strand is linked to a second terminus of the polynucleotide linker, wherein a second terminus of the displacement strand is linked to a second label, and wherein the displacement strand is at least partially complementary to a portion of the aptamer,
wherein in the concentration of a target analyte causes a shift in equilibrium between a reporting state and a non-reporting state of the aptamer switch polynucleotide, and
wherein one of the first and second labels produces a detectable readout in the reporting state of the aptamer switch polynucleotide.

2. The aptamer switch polynucleotide of claim 1, wherein in the absence of the target analyte, the displacement strand hybridizes to the portion of the aptamer.

3. The aptamer switch polynucleotide of claim 1, wherein in the presence of the target analyte, the aptamer binds to the target analyte, the displacement strand does not hybridize to the portion of the aptamer.

4. The aptamer switch polynucleotide of claim 1, wherein one of the first and second labels is a fluorophore and the other of the first and second labels is a quencher.

5 The aptamer switch polynucleotide of claim 4, wherein the first label is a fluorophore and the second label is a quencher.

6. The aptamer switch polynucleotide of claim 4, wherein the first label is a quencher and the second label is a fluorophore.

7. The aptamer switch polynucleotide of claim 4, wherein the quencher quenches the fluorescence from the fluorophore in the non-reporting state of the aptamer switch polynucleotide.

8. The aptamer switch polynucleotide of claim 4, wherein the fluorophore produces fluorescence as a detectable readout in the reporting state of the aptamer switch polynucleotide.

9. The aptamer switch polynucleotide of claim 1, wherein the polynucleotide linker is a homopolymeric polynucleotide.

10. The aptamer switch polynucleotide of claim 9, wherein the polynucleotide linker is a poly-thymine polynucleotide.

11. The aptamer switch polynucleotide of claim 1, wherein the displacement strand is between 60% and 100% complementary to the portion of the aptamer.

12. The aptamer switch polynucleotide of claim 11, wherein the displacement strand is between 80% and 100% complementary to the portion of the aptamer.

13. The aptamer switch polynucleotide of claim 12, wherein the displacement strand is between 95% and 100% complementary to the portion of the aptamer.

14. The aptamer switch polynucleotide of claim 1, wherein the displacement strand comprises 1 or 2 mismatched nucleotides.

15. The aptamer switch polynucleotide of claim 1, wherein the displacement strand is between 3 and 15 nucleotides long.

16. The aptamer switch polynucleotide of claim 1, wherein the displacement strand is at least partially complementary to an end or internal sequence of the aptamer.

17. The aptamer switch polynucleotide of claim 1, wherein the polynucleotide linker is between 10 and 100 nucleotides long.

18. The aptamer switch polynucleotide of claim 1, wherein the aptamer switch polynucleotide comprises natural and/or non-natural nucleotides.

19. The aptamer switch polynucleotide of claim 18, wherein the natural and/or non-natural nucleotides are natural and/or non-natural DNA and/or RNA nucleotides.


SEQUENCE LISTING (41 sequences)

SEQ ID NO: 1 - DNA, 53 bp, synthetic construct
  misc_feature 1..53: note = Description of Artificial Sequence: Synthetic oligonucleotide
  source 1..53: mol_type = other DNA
cacctgggggagtattgcggaggaaggttttttttttttttttcccccaggtg

SEQ ID NO: 2 - DNA, 41 bp, synthetic construct
  misc_feature 1..41: note = Description of Artificial Sequence: Synthetic oligonucleotide
  source 1..41: mol_type = other DNA
cacctgggggagtattgcggaggaaggtttttccccaggtg

SEQ ID NO: 3 - DNA, 43 bp, synthetic construct
  misc_feature 1..43: note = Description of Artificial Sequence: Synthetic oligonucleotide
  source 1..43: mol_type = other DNA
cacctgggggagtattgcggaggaaggtttttttccccaggtg

SEQ ID NO: 4 - DNA, 51 bp, synthetic construct
  misc_feature 1..51: note = Description of Artificial Sequence: Synthetic oligonucleotide
  source 1..51: mol_type = other DNA
cacctgggggagtattgcggaggaaggtttttttttttttttccccaggtg

SEQ ID NO: 5 - DNA, 54 bp, synthetic construct
  misc_feature 1..54: note = Description of Artificial Sequence: Synthetic oligonucleotide
  source 1..54: mol_type = other DNA
cacctgggggagtattgcggaggaaggttttttttttttttttttccccaggtg

SEQ ID NO: 6 - DNA, 61 bp, synthetic construct
  misc_feature 1..61: note = Description of Artificial Sequence: Synthetic oligonucleotide
  source 1..61: mol_type = other DNA
cacctgggggagtattgcggaggaaggtttttttttttttttttttttttttccccaggtg

SEQ ID NO: 7 - DNA, 39 bp, synthetic construct
  misc_feature 1..39: note = Description of Artificial Sequence: Synthetic oligonucleotide
  source 1..39: mol_type = other DNA
cacctgggggagtattgcggaggaaggttttcccaggtg

SEQ ID NO: 8 - DNA, 41 bp, synthetic construct
  misc_feature 1..41: note = Description of Artificial Sequence: Synthetic oligonucleotide
  source 1..41: mol_type = other DNA
cacctgggggagtattgcggaggaaggttttttcccaggtg

SEQ ID NO: 9 - DNA, 49 bp, synthetic construct
  misc_feature 1..49: note = Description of Artificial Sequence: Synthetic oligonucleotide
  source 1..49: mol_type = other DNA
cacctgggggagtattgcggaggaaggttttttttttttttcccaggtg

SEQ ID NO: 10 - DNA, 52 bp, synthetic construct
  misc_feature 1..52: note = Description of Artificial Sequence: Synthetic oligonucleotide
  source 1..52: mol_type = other DNA
cacctgggggagtattgcggaggaaggtttttttttttttttttcccaggtg

SEQ ID NO: 11 - DNA, 59 bp, synthetic construct
  misc_feature 1..59: note = Description of Artificial Sequence: Synthetic oligonucleotide
  source 1..59: mol_type = other DNA
cacctgggggagtattgcggaggaaggttttttttttttttttttttttttcccaggtg

SEQ ID NO: 12 - DNA, 37 bp, synthetic construct
  misc_feature 1..37: note = Description of Artificial Sequence: Synthetic oligonucleotide
  source 1..37: mol_type = other DNA
cacctgggggagtattgcggaggaaggtttccaggtg

SEQ ID NO: 13 - DNA, 39 bp, synthetic construct
  misc_feature 1..39: note = Description of Artificial Sequence: Synthetic oligonucleotide
  source 1..39: mol_type = other DNA
cacctgggggagtattgcggaggaaggtttttccaggtg

SEQ ID NO: 14 - DNA, 47 bp, synthetic construct
  misc_feature 1..47: note = Description of Artificial Sequence: Synthetic oligonucleotide
  source 1..47: mol_type = other DNA
cacctgggggagtattgcggaggaaggtttttttttttttccaggtg

SEQ ID NO: 15 - DNA, 50 bp, synthetic construct
  misc_feature 1..50: note = Description of Artificial Sequence: Synthetic oligonucleotide
  source 1..50: mol_type = other DNA
cacctgggggagtattgcggaggaaggttttttttttttttttccaggtg

SEQ ID NO: 16 - DNA, 57 bp, synthetic construct
  misc_feature 1..57: note = Description of Artificial Sequence: Synthetic oligonucleotide
  source 1..57: mol_type = other DNA
cacctgggggagtattgcggaggaaggtttttttttttttttttttttttccaggtg

SEQ ID NO: 17 - DNA, 35 bp, synthetic construct
  misc_feature 1..35: note = Description of Artificial Sequence: Synthetic oligonucleotide
  source 1..35: mol_type = other DNA
cacctgggggagtattgcggaggaaggttcaggtg

SEQ ID NO: 18 - DNA, 37 bp, synthetic construct
  misc_feature 1..37: note = Description of Artificial Sequence: Synthetic oligonucleotide
  source 1..37: mol_type = other DNA
cacctgggggagtattgcggaggaaggttttcaggtg

SEQ ID NO: 19 - DNA, 45 bp, synthetic construct
  misc_feature 1..45: note = Description of Artificial Sequence: Synthetic oligonucleotide
  source 1..45: mol_type = other DNA
cacctgggggagtattgcggaggaaggttttttttttttcaggtg

SEQ ID NO: 20 - DNA, 48 bp, synthetic construct
  misc_feature 1..48: note = Description of Artificial Sequence: Synthetic oligonucleotide
  source 1..48: mol_type = other DNA
cacctgggggagtattgcggaggaaggtttttttttttttttcaggtg

SEQ ID NO: 21 - DNA, 55 bp, synthetic construct
  misc_feature 1..55: note = Description of Artificial Sequence: Synthetic oligonucleotide
  source 1..55: mol_type = other DNA
cacctgggggagtattgcggaggaaggttttttttttttttttttttttcaggtg

SEQ ID NO: 22 - DNA, 33 bp, synthetic construct
  misc_feature 1..33: note = Description of Artificial Sequence: Synthetic oligonucleotide
  source 1..33: mol_type = other DNA
cacctgggggagtattgcggaggaaggtaggtg

SEQ ID NO: 23 - DNA, 35 bp, synthetic construct
  misc_feature 1..35: note = Description of Artificial Sequence: Synthetic oligonucleotide
  source 1..35: mol_type = other DNA
cacctgggggagtattgcggaggaaggtttaggtg

SEQ ID NO: 24 - DNA, 43 bp, synthetic construct
  misc_feature 1..43: note = Description of Artificial Sequence: Synthetic oligonucleotide
  source 1..43: mol_type = other DNA
cacctgggggagtattgcggaggaaggtttttttttttaggtg

SEQ ID NO: 25 - DNA, 46 bp, synthetic construct
  misc_feature 1..46: note = Description of Artificial Sequence: Synthetic oligonucleotide
  source 1..46: mol_type = other DNA
cacctgggggagtattgcggaggaaggttttttttttttttaggtg

SEQ ID NO: 26 - DNA, 53 bp, synthetic construct
  misc_feature 1..53: note = Description of Artificial Sequence: Synthetic oligonucleotide
  source 1..53: mol_type = other DNA
cacctgggggagtattgcggaggaaggtttttttttttttttttttttaggtg

SEQ ID NO: 27 - DNA, 53 bp, synthetic construct
  misc_feature 1..53: note = Description of Artificial Sequence: Synthetic oligonucleotide
  source 1..53: mol_type = other DNA
cacctgggggagtattgcggaggaaggttttttttttttttttcgcccaggtg

SEQ ID NO: 28 - DNA, 53 bp, synthetic construct
  misc_feature 1..53: note = Description of Artificial Sequence: Synthetic oligonucleotide
  source 1..53: mol_type = other DNA
cacctgggggagtattgcggaggaaggttttttttttttttttcccgcaggtg

SEQ ID NO: 29 - DNA, 53 bp, synthetic construct
  misc_feature 1..53: note = Description of Artificial Sequence: Synthetic oligonucleotide
  source 1..53: mol_type = other DNA
cacctgggggagtattgcggaggaaggttttttttttttttttccccctggtg

SEQ ID NO: 30 - DNA, 53 bp, synthetic construct
  misc_feature 1..53: note = Description of Artificial Sequence: Synthetic oligonucleotide
  source 1..53: mol_type = other DNA
cacctgggggagtattgcggaggaaggttttttttttttttttcccccgggtg

SEQ ID NO: 31 - DNA, 51 bp, synthetic construct
  misc_feature 1..51: note = Description of Artificial Sequence: Synthetic oligonucleotide
  source 1..51: mol_type = other DNA
cacctgggggagtattgcggaggaaggtttttttttttttttcgccaggtg

SEQ ID NO: 32 - DNA, 51 bp, synthetic construct
  misc_feature 1..51: note = Description of Artificial Sequence: Synthetic oligonucleotide
  source 1..51: mol_type = other DNA
cacctgggggagtattgcggaggaaggtttttttttttttttctccaggtg

SEQ ID NO: 33 - DNA, 51 bp, synthetic construct
  misc_feature 1..51: note = Description of Artificial Sequence: Synthetic oligonucleotide
  source 1..51: mol_type = other DNA
cacctgggggagtattgcggaggaaggtttttttttttttttcaccaggtg

SEQ ID NO: 34 - DNA, 51 bp, synthetic construct
  misc_feature 1..51: note = Description of Artificial Sequence: Synthetic oligonucleotide
  source 1..51: mol_type = other DNA
cacctgggggagtattgcggaggaaggtttttttttttttttccgcaggtg

SEQ ID NO: 35 - DNA, 51 bp, synthetic construct
  misc_feature 1..51: note = Description of Artificial Sequence: Synthetic oligonucleotide
  source 1..51: mol_type = other DNA
cacctgggggagtattgcggaggaaggtttttttttttttttcccgaggtg

SEQ ID NO: 36 - DNA, 51 bp, synthetic construct
  misc_feature 1..51: note = Description of Artificial Sequence: Synthetic oligonucleotide
  source 1..51: mol_type = other DNA
cacctgggggagtattgcggaggaaggtttttttttttttttccctaggtg

SEQ ID NO: 37 - DNA, 51 bp, synthetic construct
  misc_feature 1..51: note = Description of Artificial Sequence: Synthetic oligonucleotide
  source 1..51: mol_type = other DNA
cacctgggggagtattgcggaggaaggtttttttttttttttcccaaggtg

SEQ ID NO: 38 - DNA, 49 bp, synthetic construct
  misc_feature 1..49: note = Description of Artificial Sequence: Synthetic oligonucleotide
  source 1..49: mol_type = other DNA
cacctgggggagtattgcggaggaaggttttttttttttttcgcaggtg

SEQ ID NO: 39 - DNA, 49 bp, synthetic construct
  misc_feature 1..49: note = Description of Artificial Sequence: Synthetic oligonucleotide
  source 1..49: mol_type = other DNA
cacctgggggagtattgcggaggaaggttttttttttttttccgaggtg

SEQ ID NO: 40 - DNA, 49 bp, synthetic construct
  misc_feature 1..49: note = Description of Artificial Sequence: Synthetic oligonucleotide
  source 1..49: mol_type = other DNA
cacctgggggagtattgcggaggaaggttttttttttttttccctggtg

SEQ ID NO: 41 - DNA, 49 bp, synthetic construct
  misc_feature 1..49: note = Description of Artificial Sequence: Synthetic oligonucleotide
  source 1..49: mol_type = other DNA
cacctgggggagtattgcggaggaaggttttttttttttttcccacgtg